(19) United States
(12) Patent Application Publication
(10) Pub. No.: US 2022/0319659 A1
(43) Pub. Date: Oct. 6, 2022

(54) DEVICES AND METHODS FOR SECURITY AND TRACKING OF ITEMS

(71) Applicant: MIDAS Healthcare Solutions, Inc., Center Moriches, NY (US)

(72) Inventors: Michael A. LAFAUCI, Center Moriches, NY (US); Jeffrey R. WAHL, Beachwood, OH (US); Jonathan PINSKY, Bedford, NY (US)

(21) Appl. No.: 17/571,063

(22) Filed: Jan. 7, 2022

Related U.S. Application Data
(63) Continuation of application No. PCT/US20/41257, filed on Jul. 8, 2020.
(60) Provisional application No. 62/872,176, filed on Jul. 9, 2019.
(60) Provisional application No. 62/873,614, filed on Jul. 12, 2019.

(51) Int. Cl. G16H 20/13; G16H 40/67; B65D 83/04; G07C 9/00

(52) U.S. Cl. CPC G16H 20/13; G16H 40/67; B65D 83/04; G07C 9/00174

(57) ABSTRACT

The present disclosure provides systems and methods for secure retrieval, tracking, administration, and/or return of contents such as oral medications. In some cases, a system for securing and tracking contents within a container is provided. The system may comprise an adapter configured to couple to an opening to the container, or to a cap of the container. The adapter can be configured to operate and switch between an unlocked state and a locked state. The system may also comprise a key device paired with the adapter. The key device can be configured to cause the adapter to switch from the locked state to the unlocked state upon activation of the key device, thereby permitting a user to open the container and access the contents from the container.

### CROSS-REFERENCE

This application is a continuation of International Patent Application No. PCT/US20/41257, filed Jul. 8, 2020, which claims the benefit of U.S. Provisional Patent Application No. 62/872,176, filed Jul. 9, 2019, and U.S. Provisional Patent Application No. 62/873,614, filed Jul. 12, 2019, each of which is entirely incorporated herein by reference.

### BACKGROUND

Diversion of prescription medications (e.g., controlled substances such as opioids and other pharmaceuticals) for illicit purposes, unintended uses and accidental exposures can lead to medical issues (e.g. fetal exposure from maternal substance uses; a minor finding and accidentally ingesting adult-strength prescription medication or medication intended only for adults, that can be stored in various places such as a medicine cabinet, a drawer at home, in a parent's handbag, etc., leading to unintended health consequences for the minor), hospitalization or even death. The prescription medications can be manufactured, dispensed, delivered, applied, and/or administered in multiple forms, including oral drugs, injectable drugs, and transdermal patches. The diversion of prescription medications can account for about 30 percent (%) of the overall drug problem in the United States. Furthermore, the diversion of prescription medications can cost billions of dollars of economic burden in areas including law enforcement, health care, social services, and court costs. In some cases, opioid diversion through theft or unlawful distribution can cost over $70 billion to insurers and employers. In such cases, the employers can be adversely affected by lack of productivity, absence and/or carelessness of employees with substance abuse issues. More importantly, there is a substantial risk of adverse health consequences (leading possibly to bodily harm or death) to patients whose medications have been diverted by caregivers or any other unauthorized third party for personal use or illicit financial gains, such as the illegal sale or distribution of those medication. By depriving patients of the necessary medications ordered for them, serious harm (from a health standpoint as well as financially) can be inflicted on those patients.

The diversion of prescription medications can be related to lack of compliance by patients, family members, caregivers, and/or medical practitioners (e.g., healthcare providers), and also the intentional illicit behavior involving the diversion or misuse of such medications by certain personnel working in the healthcare profession, or by third parties having access to those medications. In some cases, approximately 15% of pharmacists, approximately 10% of nurses and/or approximately 8% of physicians can be involved in the diversion and/or illicit distribution of prescription medications for non-prescription (e.g., recreational) uses. Practitioners with knowledge and access to such prescription medications can tamper with their storage means (e.g., containers or vessels) or treatment vehicles (e.g., transdermal patches) to extract, use, and/or distribute residual pharmaceuticals.

There is currently a lack of security for many prescription medication containers (e.g. Rx bottles), and lack of ways to track or monitor patient compliance. Given that many existing medication containers are not secured and tracked, it often gives rise to diversion or misuse as described above.

### SUMMARY

There is a need for improved security, transport, and/or tracking of certain contents (e.g., prescription medications, confidential items, etc.). For example, there is a need for improved security, transport, and/or tracking of certain medications (e.g., controlled drugs, non-controlled drugs, over-the-counter drugs, prescription drugs, non-prescription drugs, etc.) pre-administration, during administration, post-administration, and pre-destruction (e.g., prior to the destruction of unused, leftover, refused, misplaced medications). In another example, the contents can be components or starting materials of drugs, food, or other manufactured products.

The devices and methods disclosed herein can be configured to contain and secure medications (e.g., oral medications, injectable medications, etc.) in both outpatient and institutional environments, for example at a subject's home, office, public places, and hospitals. The present disclosure provides devices and methods for securing a packaging that is used to contain the medications for patients, and to track the removal of the medications each time the packaging is being opened, closed, locked, unlocked, or tampered with. The packaging can include a variety of containers of different shapes and/or sizes for holding different types of the medications. Examples of the oral medications can include tablets, caplets, liquid capsules, liquid medications, and the like. Examples of the injectable medications can include liquid medications for, e.g., intravenous or intramuscular injections. Examples of containers may include pharmacy bottles, pill containers, vials, medicine bottles, and other pharmacy supplies for storing prescription medications.

The devices and methods described herein can permit tracking of contents (e.g. oral prescription medications), and detect if a patient is administered (e.g., consuming) the medication at a preset schedule, poor adherence or lack of compliance, whether the medication is being misused or diverted, whether the medication is lost or stolen, etc. In some cases, the devices and methods can also be used to track the container and its contents (e.g., the medications) as they are being transported from a patient's home to a target location. The target location can be a take-back system at a pharmacy, a return facility for destruction of unused medication, the patient's healthcare provider at a hospital or clinic, etc.

Accordingly, the devices and methods herein can be used for the secure handling and tracking of the contents of the container (e.g. oral prescription medications) at the patient's home/office, within a healthcare institution, intra or between institutions, between an institution and an outpatient retail pharmacy, between the institution and the patient's home, between the outpatient retail pharmacy and the patient's home, between the institution/retail pharmacy/patient's home and one or more intermediary locations, between the institution/retail pharmacy/patient's home and any third parties that manufacture, ship, collect, and/or dispose of (new or used) medications, etc.

An aspect of the present disclosure provides a system for securing and tracking contents within a container, the system comprising: an adapter configured to couple to an opening of the container or a cap of the container, wherein the adapter is configured to operate and switch between an unlocked state and a locked state; and a key device paired with the adapter, wherein the key device is configured to cause the adapter to switch from the locked state to the unlocked state upon activation of the key device, thereby permitting a user to open the container and access the contents from the container.

In some embodiments, the user is a patient or a caregiver for the patient, and the contents include oral medications. In some embodiments, the key device and the adapter are associated with the patient or the caregiver.

In some embodiments of any of the systems herein, the key device is activated when the adapter is brought in proximity to the key device. In some embodiments, the key device and the adapter are in wireless communication with each other when the adapter is brought in proximity to the key device.

In some embodiments of any of the systems herein, the adapter and/or the container is configured to display (i) a first scannable code when the contents are secured in the locked container and/or (ii) a second scannable code when the container is unlocked and the contents accessed by the user.

Another aspect of the present disclosure provides a method for securing and tracking contents within a container, the method comprising: (a) providing (i) an adapter configured to operate and switch between an unlocked state and a locked state and (ii) a key device paired with the adapter; (b) coupling the adapter to an opening of the container or a cap of the container; and (c) subsequent to (b), activating the key device to cause the adapter to switch from the locked state to the unlocked state, thereby permitting a user to open the container and access the contents from the container.

In some embodiments, the user is a patient or a caregiver for the patient, and the contents include oral medications. In some embodiments, the key device and the adapter are associated with the patient or the caregiver.

In some embodiments of any of the methods herein, the key device is activated when the adapter is brought in proximity to the key device. In some embodiments, the key device and the adapter are in wireless communication with each other when the adapter is brought in proximity to the key device.

In some embodiments of any of the methods herein, the adapter and/or the container is configured to display (i) a first scannable code when the contents are secured in the locked container and/or (ii) a second scannable code when the container is unlocked and the contents accessed by the user.

Another aspect of the present disclosure provides a system for securing and tracking contents in a package, the system comprising: a housing configured to contain the package comprising the contents; a sensor unit operatively coupled to the housing, wherein the sensor unit is configured to detect removal of at least a portion of the contents from the package when the package is contained within the housing; and a controller operatively coupled to the sensor unit, wherein the controller is configured to direct the sensor unit to detect the removal of the at least the portion of the contents from the package within the housing.

In some embodiments, the sensor unit is contained within the housing.

In some embodiments of any of the systems herein, the contents comprises a plurality of items and the sensor unit comprises a plurality of sensors, wherein a sensor of the plurality of sensors is configured to detect removal of an item of the plurality of items.

In some embodiments of any of the systems herein, the contents comprise medications. In some embodiments, the medications comprise oral medications.

In some embodiments of any of the systems herein, the package is a blister package comprising a plurality of oral medications.

In some embodiments of any of the systems herein, (i) the housing is configured to operate and switch between an unlocked state and a locked state, and (ii) the controller is operatively coupled to the housing and configured to determine the switching of the housing between the unlocked state and the locked state.

Another aspect of the present disclosure provides a method for securing and tracking contents in a package, the method comprising: (a) providing (i) a housing and (ii) a sensor unit operatively coupled to the housing; (b) using the housing to contain the package comprising the contents within the housing; and (c) using the sensor unit to detect removal of at least a portion of the contents from the package when the package is contained within the housing.

In some embodiments, the sensor unit is contained within the housing.

In some embodiments of any of the methods herein, the contents comprises a plurality of items and the sensor unit comprises a plurality of sensors, wherein a sensor of the plurality of sensors is configured to detect removal of an item of the plurality of items.

In some embodiments of any of the methods herein, the contents comprise medications. In some embodiments, the medications comprise oral medications.

In some embodiments of any of the methods herein, the package is a blister package comprising oral medications.

In some embodiments of any of the methods herein, the housing is configured to operate and switch between an unlocked state and a locked state, and the method further comprises determining the switching of the housing between the unlocked state and the locked state.

Another aspect of the present disclosure provides a system for secure transport of contents, the system comprising: a carrier configured to receive and securely store the contents therein, wherein the carrier is configured to operate and switch between an unlocked state and a locked state, and wherein the carrier in the locked state is used by a user to transport the contents from a first location to a second location; and a key device paired with the carrier, wherein the key device is configured to cause the carrier to switch from the locked state to the unlocked state upon activation of the key device, thereby permitting the user to access the contents from the carrier at the second location.

In some embodiments, the user is a healthcare practitioner, and the contents include medications or medical devices/equipment. In some embodiments, the contents are administered by the healthcare practitioner to a patient at the second location.

In some embodiments of any of the systems herein, the key device is provided at the second location, wherein the key device is activated when the carrier is brought in proximity to the key device at the second location. In some embodiments, the key device is associated with a patient at the second location.

In some embodiments of any of the systems herein, the key device is provided at a third location remote to the second location, wherein the key device is used to remotely unlock the carrier at the second location. In some embodiments, the key device is associated with a third party at the third location, wherein the third party is an advocate or proxy for the patient authorized to approve certain medical healthcare decisions for the patient.

In some embodiments of any of the systems herein, the key device and the carrier are in wireless communication with each other.

In some embodiments of any of the systems herein, the carrier is configured to display (i) a first code when the contents are secured in the locked carrier at the first location, wherein the first code is configured to be scanned by the user at the first location, and/or (ii) a second code when the carrier is unlocked at the second location and the contents accessed by the user, wherein the second code is configured to be scanned by the user at the second location.

Another aspect of the present disclosure provides a method for securing transport of contents, the method comprising: (a) providing (i) a carrier configured to operate and switch between an unlocked state and a locked state, wherein the carrier in the locked state is used by a user to transport the contents from a first location to a second location and (ii) a key device paired with the carrier; (b) using the carrier to receive and securely store the contents therein; (c) subsequent to (b), activating the key device to cause the carrier to switch from the locked state to the unlocked state, thereby permitting the user to access the contents from the carrier at the second location.

In some embodiments of any of the systems herein, the user is a healthcare practitioner, and the contents include medications or medical devices/equipment. In some embodiments, the contents are administered by the healthcare practitioner to a patient at the second location.

In some embodiments of any of the systems herein, the key device is provided at the second location, and the method further comprises activating the key device by bringing the carrier in proximity to the key device at the second location. In some embodiments, the key device is associated with a patient at the second location.

In some embodiments of any of the systems herein, the key device is provided at a third location remote to the second location, wherein the key device is used to remotely unlock the carrier at the second location. In some embodiments, the key device is associated with a third party at the third location, wherein the third party is an advocate or proxy for the patient authorized to approve certain medical healthcare decisions for the patient.

In some embodiments of any of the systems herein, the key device and the carrier are in wireless communication with each other.

In some embodiments of any of the systems herein, the carrier is configured to display (i) a first code when the contents are secured in the locked carrier at the first location, wherein the first code is configured to be scanned by the user at the first location, and/or (ii) a second code when the carrier is unlocked at the second location and the contents accessed by the user, wherein the second code is configured to be scanned by the user at the second location.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a schematic view of an adapter coupled to an oral medication container, along with a key device for unlocking the adapter, in accordance with some embodiments;

FIG. 2 shows the securing of the container using the adapter of FIG. 1, and the display of a unique code upon securing the container, in accordance with some embodiments;

FIGS. 3A-3E show the unlocking and securing of the oral medication container using the adapter, in accordance with some embodiments;

FIG. 4 shows an adapter coupled to an oral medication container, in accordance with some embodiments;

FIG. 5 shows an exemplary flowchart of the unlocking and securing of the oral medication container using the adapter of FIG. 4;

FIG. 6A shows a prescription programmed box for containing medications, in accordance with some embodiments;

FIG. 6B shows different schematic views of the prescription programmed box, in accordance with some embodiments;

FIG. 7 shows an exemplary flowchart of using the prescription programmed box, in accordance with some embodiments;

FIG. 8 shows schematic view of a secure transport system comprising at least one carrier and at least one key device, in accordance with some embodiments;

FIGS. 9A and 9B show examples of a carrier paired with a key device, in accordance with some embodiments;

FIGS. 10A and 10B show the unlocking of a locked carrier when the carrier is placed in proximity to the key device;

FIG. 11 shows a container for distribution and delivery of items to a destination; and

FIG. 12 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “at most”, “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### I. Overview

The devices and methods described herein may be used anywhere as a self-containing security device to secure and track prescription medication (e.g. oral medication). Any type of oral medications can be secured and tracked using the devices and methods disclosed herein. The oral medications may include controlled substances, non-controlled substances, high-cost drugs or therapeutics, etc. In some cases, the oral medications may have high value, and may be subject to high risks of diversion or theft if they are stored in unsecured containers. Accordingly, the devices and methods described herein can help to reduce or eliminate the risk of diversion or misuse of medications (e.g., oral medications) at the patient's home, at a healthcare institution, or any physical location. The devices can be used to secure an oral medication from the time and point of dispensing, to the time and point when the patient is about to take the oral medication. The devices may also serve as a self-contained security device for medications to be accounted for during their wasting process.

Examples of a healthcare institution can include hospitals (e.g., medication room, patient room, surgery room, etc.), medical offices (e.g., physician clinics, dental clinics, ambulatory surgery centers, same-day or other non-hospital surgery facilities, etc.), emergency response units (e.g., paramedic transportations, emergency medical service (EMS) transportations, etc.) for transporting patients, veterinary hospitals, veterinary clinics, veterinary laboratories, medical research facilities, hospice, long-term acute care (LTAC) facility, nursing home, assisted living facility, pharmacy, in-pharmacy clinic, etc.

The medications, as provided herein, may or may not require prescription (e.g., by healthcare professionals, such as physicians). In some examples, prescriptions are not needed for over-the-counter medications, such as, for example, Robitussin, Tylenol, and Sudafed. The medications, as provided here, may or may not be controlled. Examples of non-controlled prescription substances include antibiotics, cholesterol medication, and Viagra.

Examples of controlled substances can comprise opiate and opioids, as well as central nervous system (CNS) depressants and stimulants. Examples of opioids can include morphine, codeine, thebaine, oripavine, morphine dipropionate, morphine dinicotinate, dihydrocodeine, buprenorphine, etorphine, hydrocodone, hydromorphone, oxycodone, oxymorphone, fentanyl, alpha-methylfentantyl, alfentanyl, trefantinil, brifentanil, remifentanil, octfentanil, sufentanil, carfentanyl, meperidine, prodine, promedol, propoxyphene, dextropropoxyphene, methadone, diphenoxylate, dezocine, pentazocine, phenazocine, butorphanol, nalbuphine, levorphanol, levomethorphan, tramadol, tapentadol, anileridine, any functional variant thereof, or any functional combinations thereof. Examples of CNS depressants and stimulants can include methylphenobarbital, pentobarbital, diazepam, clonazepam, chlordiazepoxide, alprazolam, triazolam, estazolam, any functional variant thereof, or any functional combinations thereof.

Additional examples of the medications and the relevant therapeutic applications include scopolamine for motion sickness, nitroglycerin for angina, clonidine for hypertension, and estradiol for female hormone replacement therapy. Other examples of the drugs include, but are not limited to, methylphenidate, selegiline, rivastigmine, rotigotine, granisteron, buprenorphine, estradiol, fentanyl, nicotine, testosterone, propofol, etc.

### II. Container and Adapter

A. Medication Container

The devices described herein can utilize or comprise an adapter for securing an opening of a medication container. The container can be used for storing any type of medication (e.g., oral medications) as described herein. The container may be formed having any shape, design, and/or size. The adapter can be used to secure the openings of a variety of different types of containers. For example, the adapter may include an adjustable ring or sleeve that can be adjusted to fit a container opening of any size. The shape, design, and/or size of the container may depend on the contents to be transported and secured (e.g. size, volume, mass, shape, and/or quantity of the medications). The container may have any regular shape or irregular shape. Examples of possible shapes or designs may include but are not limited to: mathematical shapes, two-dimensional geometric shapes, multi-dimensional geometric shapes, curves, polygons, polyhedral, polytopes, minimal surfaces, ruled surfaces, non-orientable surfaces, quadrics, pseudospherical surfaces, algebraic surfaces, miscellaneous surfaces, riemann surfaces, box-drawing characters, cuisenaire rods, geometric shapes, shapes with metaphorical names, symbols, unicode geometric shapes, or any other form. In some embodiments, the container may have a substantially cylindrical, round/circular, square, rectangular, oval, triangular, or elliptical shape. A container as described herein may comprise a single hollow interior or cavity. In some embodiments, a container may comprise a plurality of compartments, chambers, pockets, or sleeves for holding a same type of medication, or different types of medications.

The container can include a hollow interior for storing contents (e.g. medications, such as oral medications). The container has at least one opening for receiving contents. The container may be provided with a pre-existing cap. In other embodiments, the container need not be provided with a pre-existing cap. Access to the interior of the container can be regulated/controlled using the adapter which is either coupled directly to the opening of the container, or coupled to the pre-existing cap of the container. The container can be configured to switch between an unlocked state and a locked state, by unlocking or locking the adapter at the opening of the container. Access to the interior of the container can be achieved by manipulating the adapter, for example by twisting, rotating, sliding, pushing, pulling, pressing, lifting, pinching, or grasping the adapter. The adapter may include a locking mechanism for securing the container, such that the contents are not accessible by an unauthorized party when the container is locked by the adapter.

The adapter can be used to lock or unlock the container any number of times. The locking and unlocking may be location dependent. For example, the adapter can lock the container after the container is secured with new medication pre-administration, or is stored with unused medication post-administration. A patient can unlock the adapter to open the container when the patient is about to take the medication (or when the medication is about to be administered to the patient). The container can also be locked by the adapter during the transportation of any unused or wasted medication. The container can be unlocked at the return or take-back system when the unused medication is returned for wasting/destruction. The locking and unlocking of the container, at different instances in time corresponding to different physical locations and different events, may trigger different codes indicating which action is being performed at the corresponding juncture (e.g. consumption of medication by patient, administering of the medication to the patient by a caregiver, possible misuse of the medication, potential diversion, etc.). The codes can be used to track the dispense and handling of medication, as well as the return of unused medication, in a closed loop real-time secure tracking process. The adapter and container are designed to be tamper-proof. Any unauthorized attempts to tamper, break into, open or hack the adapter/container to obtain the medication from the container in an illicit behavior can result in notifications and/or alerts being sent, informing the relevant authorities that the container and its contents have been compromised. In some cases, the locking and unlocking of the container by the adapter may not and need not be location dependent.

In some embodiments, a key device may be provided to the patient (or to the patient's caregiver), in association with the adapter and the container containing the medications for the designated patient. The key device may be specific or unique to that patient (or for the patient's caregiver). The key device may be provided in various configurations or forms, for example as a wearable that is worn on the patient's body or caregiver's body (e.g. wrist or other extremity), etc. In some embodiments, the key device may be provided as a software/app on a wearable device (e.g. smartwatch) or mobile device (e.g. smartphone). The key device and the adapter can be paired with each other, such that a specific assigned key device is used to unlock the adapter for a container for a designated patient. The key device can be configured to wirelessly communicate with the adapter to unlock the adapter, when both are brought into proximity with each other near the patient (e.g. via Bluetooth, NFC). Optionally, the adapter can be unlocked (e.g. via a telecommunications network or via a secure connection over the Internet) by a key device located with the patient's caregiver. Accordingly, the adapter can only be opened by the assigned patient's key device, or when unlocked by a key device located with the patient's caregiver. This can significantly reduce or eliminate the risk of diversion as the medication is being dispensed from the container to the patient.

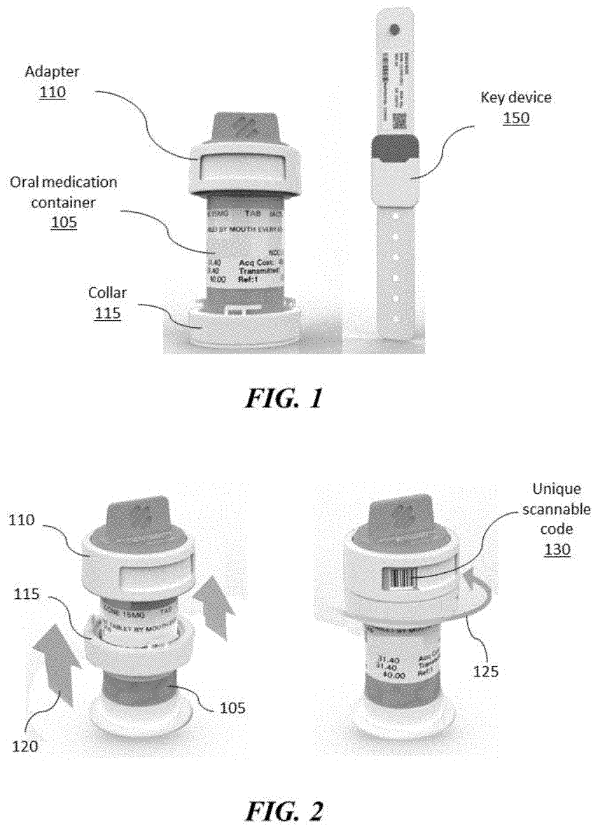

Referring to FIG. 1, an adapter 110 is shown coupled to an oral medication container 105. In some embodiments, the adapter can be used as a cap/lid for the container. In other cases, the adapter can be configured to attach or couple to a pre-existing cap of the container. The adapter can be used to secure oral medication for safe handling/proper dispense, and for the return of unused medication. The adapter can be used to track unused medication, and can be configured for use with a return or take-back system. Examples of take-back systems or return systems that can be used with embodiments of the present disclosure are described in International Application No. PCT/US2020/026434 (titled “Systems and Methods for Medication Management”). The adapter can prevent unintended exposure and diversion of oral medication from the container. The oral medication may be initially dispensed into the container (e.g. by a pharmacist or a robot), and the container can be subsequently secured and locked using the adapter coupled to the opening of the container. In some embodiments, the container or adapter may include a RFID chip to help identify contents within the container. In some embodiments, the container or adapter may include a position sensor for detecting the physical location of the container substantially in real-time.

The container can be formed having any shape and/or size, to accommodate any number/size/shape of pills, capsules, tablets, caplets, etc. The container can be opaque, clear, or have any color, as long as the contents and quantity can be accounted for either within or outside of the container. In some embodiments, a container may be made from light resistant amber plastic, and may conform to USP light standards for light transmission and USP tight standards to protect the medication contents from degradation or contamination. The container may have a prescription label (or any other patient identifier) affixed thereto. The container can be made of any material, e.g. plastic, paper, cardboard, glass, etc. This label can point to, or identify the prescription contents for a patient. In some embodiments, the container may be flexible, or capable of folding or scalable in size to accommodate its contents and different shipping return options. For example, the container secured with contents can be returned to a take-back system either in person or by mail, Fedex, UPS, or some other expedited carrier.

FIG. 2 shows the securing of the container 105 using the adapter 110. In some embodiments, after the patient has consumed the oral medication, a collar 115 at the bottom of the container can be raised (e.g., along a direction as indicated by the arrow(s) 120) to secure the container. The collar may be located near the bottom of the container when the adapter is unlocked. The container can be locked and secured by the patient or the patient's caregiver. The collar can be configured to slide up the container, and engage with the adapter located at the opening of the container. When the collar is twisted/rotated relative to the adapter (e.g., along a direction as indicated by the arrow 125), a locking mechanism is activated to lock the adapter, that prevents the container from being opened.

Once the adapter/container has been locked, a unique scannable code 130 is revealed in a window of the adapter. In some cases, the code may be displayed on the container. The code may be activated by a code trigger mechanism on the adapter, that is associated with the locking of the adapter. The code trigger mechanism can be activated by twisting the adapter as described above, or by pushing a lever, pressing a button, pressing anywhere on the adapter, fingerprint recognition, facial recognition, eye scans, app activation, or any other mechanism which can secure the container and create a unique code/identifier. In some embodiments, the code may be a reconstructable code, for example as described in International Application No. PCT/US2020/019122 (titled “Reconstructed Segmented Codes and Methods of Using the Same”). In one example, when the adapter is twisted to a full locked orientation/configuration, one or more code segments may be pieced together in a window of the adapter to reconstitute and display a complete and unique scannable visual code.

The locking of the adapter to secure the container can trigger the unique code, which is specific to the patient and prescribed medication. The code may be displayed on any portion of the adapter, for example on the sides, top, center, partially or entirely on the adapter. In some embodiments, the sides of the adapter may include a slot, a series of slots or any other openings/recess, that permit the code to be displayed when the adapter is locked and when the code trigger mechanism is activated. The code may be obscured from view (e.g. hidden behind the slots) when the code trigger mechanism has yet to be activated.

In some cases, a patient may be unable to physically open the medication container (for example, if the patient has undergone surgery on the hand/arm, physically or mentally incapacitated, has undergone a stroke, etc.). In those cases, a key device (e.g., a key device 150 as shown in FIG. 1) may be provided to a caregiver for the patient. The key device may be provided as a discrete stand-alone device to the caregiver. In some embodiments, the key device may be provided on a mobile device (e.g. smartphone) or a wearable device (e.g. smartphone) of the caregiver. The caregiver can use his/her key device to unlock the container and administer the oral medication to the patient.

The adapter on a medication container can communicate with a key device located with the patient (or a key device located with the patient's caregiver). The communication may be wireless or wired, and can be enabled using one or more radio communication chips. Wireless communications may include Bluetooth, near-field communication (NFC), Wi-Fi, Wi-LAN, radio-frequency identification (RFID), infrared (IR), etc. Upon pairing of the key device and the adapter, the adapter can be unlocked to allow the container to be opened, and its contents (e.g. oral medication) accessible for consumption by the patient. In some embodiments, the key device 150 may be provided as a wearable (e.g. a wrist-strap) for example as shown in FIG. 1. The wearable key device can be worn by the patient or the caregiver. The key device may be located anywhere, for example on the patient (e.g. a Patient Identity (ID) Bracelet), near the patient, patient's mobile device (e.g. smartphone) or wearable device (smartwatch), etc. In some embodiments, the key device may be located remote to the patient, for example with the caregiver for the patient. As previously described, the key device may be provided on a mobile device and/or wearable device of the caregiver. The key device may be provided in the form of an app loaded on the mobile device or wearable device. The key device may require the caregiver to enter a password to unlock the adapter. Alternatively, the key device may require the caregiver's biometric identification (e.g. fingerprints, iris scans, facial recognition scans, etc.) to unlock the adapter. In some cases, a code may be sent to the caregiver's mobile device or wearable device, and the caregiver has to enter the code into the app to unlock the adapter.

Each adapter and each key device for an oral medication container for a patient may be provided with its own unique code (or identifier). The code on an assigned key device can be designed to match or pair with the code on the designated adapter, in order to unlock the adapter and open the container to retrieve its oral medications. Examples of codes or identifiers that can be used with embodiments of the present disclosure are described in International Application No. PCT/US2020/019122 (titled “Reconstructed Segmented Codes and Methods of Using the Same”), and International Application No. PCT/US2019/042059 (titled “Systems and Methods for Removing a Covering from a Bodily Surface”).

Oral medication can be loaded into an empty container and locked using the adapter. This can occur at a retail pharmacy, on site or near to an ADM, in a medication room, or elsewhere in accordance with policy and procedure. Once the adapter and container are locked, a tracking system can generate a code, which would be unique to that specific medication and to a patient, with a timestamp indicating that a locked oral medication is now contained within the container. The code may include or utilize any of the codes described elsewhere herein. A pharmacist or nurse may scan the code which may subsequently send an alert/signal to a central tracking system (and/or EMR, Pyxis, etc.) that signifies the oral medication container has been secured with the corresponding contents, and that the container is now ready for pick up by the patient or the patient's caregiver.

The patient or the patient's caregiver may require the key device for the specific oral medication container/adapter, in order to pick up the medication. After the container has been picked up, the container with its contents may be unlocked at different locations and at different points in time for ingestion by the patient.

The key device can be used to unlock the adapter once communications have been established, thereby allowing the patient or caregiver to retrieve the oral medication(s) from the container. The container/adapter can be unlocked using signals transmitted wirelessly (e.g. via Bluetooth, RFID, Near Field Communication (NFC), Wi-Fi, Infrared (IR), etc.) between the key device and adapter. In some embodiments, the adapter and the key device may utilize a reconstructable visual code. The reconstructable visual code may be segmented, and the different visual code segments may be provided to a paired adapter and key device. For example, a first visual code segment may be displayed on the adapter, and a second visual code segment may be displayed on the key device when the adapter is brought in proximity to the key device. The patient (or caregiver) may place the key device adjacent to the adapter to effectively reconstruct the unique pattern of the visual code. The unique pattern may be detectable by a visual detecting device (e.g., a mobile scanner). Upon reconstruction of the visual code, the adapter and container can be unlocked, thereby permitting the patient or caregiver to access the oral medication(s).

The adapter can be unlocked, and the container can be opened using the key device. In some embodiments, once the adapter and the container are opened, a unique code may be triggered appearing on the adapter and/or the container. The patient or caregiver may be required to scan the code when the code appears, and after the patient has consumed the oral medication. Scanning of the code collects information indicating when the container is opened by the patient or caregiver, for the designated drug, at a given instance in time.

In some instances, if the patient is unable to complete all of the prescribed medication, the patient or caregiver may use the same container with adapter to store and secure/re-lock the unused medication. The container containing the unused medication may be returned to a take-back system for destruction. In some embodiments, returned medications in a container can be identified using different color codes, or other types of codes or unique identifiers that are triggered for certain events, for example when the patient refuses to take the medication and/or if the patient is unable to complete the full course of medication. The color codes or other unique identifiers may be triggered electronically by activating a switch on the adapter or container. Alternatively, the color codes or other unique identifiers may be triggered manually, for example by the user (e.g. patient or caregiver) sliding, pulling, or physically activating a mechanism which causes the specific code to appear, or which causes a change in an original color code of the container or adapter. As an example, when the patient refuses the medication, a color code on the adapter or container may switch from a first color code (green) to a second color code (e.g. red). In another instance, if the medication is not completely administered and the container is used to return the residual medication, the color code on the adapter or container may switch from the first color code (green) to a third color code (e.g. blue).

A unique code can be used to link a prescription order to the adapter and container for return. After the patient or caregiver loads and locks the unused medication in the container using the adapter, the patient or caregiver may scan the code using a scanner, which may then trigger or create an assignment for the waste process for that unused medication and designated patient with the locked container. Once activated, the code can be scanned into a tracking system, thereby linking the patient/caregiver, and the unused medication to be wasted. Additionally, the information can be linked to a proxy or advocate for the patient, so as to alert the advocate about the latest and relevant events (e.g. medication successfully administered, or patient refused the medication, etc.). This can provide real-time updates and timely awareness to the advocate about the patient's care/status.

In some embodiments, when the container containing the unused/wasted medication is scanned, an image of the unused medication inside of the locked container may be captured by the return system. The image can be linked with the unused/wasted medication, designated patient and specific container/adapter. The images and containers can be incorporated into an intelligent medication return or take-back system, for example as described in U.S. 62/830,215 as mentioned above. The devices described herein can also be utilized as an integral component of the wasting process for oral medications.

As described herein, a series of discrete events, starting (1) from the dispense process of the oral medication into a container for a particular patient, (2) the administration of the medication by the patient or by the caregiver, and (3) the secure return or wasting of unused medication, is a closed loop tracking process containing various checkpoints and safeguards. This ensures a high level of security and accountability for oral medications, which can reduce or eliminate the risk of diversion.

Each of the key device, adapter, and/or container may be disposable or non-disposable. Each of the key device, adapter, and container may be configured for multiple use, or for single one-time use. Each of the key device, adapter, and/or container may be made of a variety of materials, for example plastics, vinyl, cardboard etc. Unique codes for the key device, adapter, and/or container may be displayed on any portion thereof.

In some embodiments, the container may include a cap that can slide, flip, or rotate open (or close). The cap may be the adapter. Alternatively, the adapter may include or incorporate the cap. The cap may include a display window or panel. In some embodiments, a unique code for the container (when it is locked and holding an oral medication within), may appear in the display window or panel. In other embodiments, the display window or panel need not be on the cap, and may be provided anywhere on the container, for example on a front or back surface of the container.

When the system (comprising the container/adapter/key device) is activated/locked/opened/used, signals can be sent to a tracking system confirming the locking/use/opening of the container and the functions (e.g. retrieval, drug administering, wasting, etc.) that are being performed. Accordingly, the system serves as a closed loop tracking system, and can help to verify/confirm/ensure that the oral medication secured in the container is timely and appropriately consumed by the patient. The disclosed system and its various checkpoints and safeguards can help to deter, reduce or eliminate potential diversion behavior, which is a systemic issue that current protocols (e.g. those based on an “honor” system) are unable to rectify.

The medication container with adapter can be used with an intelligent wasting system. A unique code on the secured and loaded container can be scanned to link the information of the waste medication to the intelligent wasting system. Additionally, images of the contents (e.g., syringe, vial) within the container can be captured for visual confirmation and verification. The container can then be placed into the appropriate section of the intelligent wasting system for destruction by a DEA reverse collector.

The individual code for each adapter, container and/or key can be reset to enable multiple, sequential uses of adapters/containers with keys. The codes can also be reset when the adapter is used for another container containing a different medication, or when the adapter/key device is assigned to a different patient and caregiver. The adapters, containers and/or keys may be reset, paired, assigned, or synchronized in any combination to support usage with a large number of patients and caregivers in outpatient environments.

The devices and methods described herein can also be applied to outpatient environments. For example, the devices can be used with dispense of medication at the retail level (e.g. an outpatient pharmacy), secure transport, and access to the medication at the patient's home or other non-institutional locations.

A container may be initially provided at or to the pharmacy. The container can contain the dispensed medication(s). Alternatively, the container may not and need not contain the dispensed medication(s) initially. The container may be activated and locked/unlocked at the time of dispensing, using for example the adapter described herein. The container, adapter and key device may be matched to the specific pharmacist, patient, medication, at a given point in time. The unique code generated by a locked container can be scanned at the retail/outpatient dispensing level, thereby linking the above information.

When the patient or caregiver (or proxy) is picking up the container containing the dispensed oral medication, the patient or caregiver may show a code for pickup that correlates with the locked container. The code may be provided on a key device held by the patient or caregiver. In some embodiments, the key device may be implemented on a mobile device or wearable device of the patient or caregiver. When the pharmacist scans the pickup code, a signal is generated indicating that the medication secured within the container by the adapter has been picked up. The tracking of the container may be monitored using a combination of software and hardware at the retail drug store level, and/or at designated patient or caregiver level.

In some cases, an employee (e.g., a clerk) of a pharmacy (e.g., a retail pharmacy) may scan a code (e.g., a scannable code provided on a receipt) to retrieve the prescribed medications that are ready for pickup. The prescribed medications may be prepared (e.g., packaged) in one or more bags, and the bag(s) may be placed on a shelf or within a bin at the pharmacy. Upon scanning the code and retrieving information about the prescribed medications (e.g., identification number, and/or location of the prescribed medications at the pharmacy), the employee may retrieve the prescribed medications (e.g., from the shelf) and provide them to the patient or the caregiver. Such process of distribution of prescribed medications can be improved (e.g., security enhanced, diversion reduced, etc.) by use of any device disclosed herein. In an example, the prescribed medications may be stored in a secure container, such as a security locker that comprises a plurality of lockers for securely storing a plurality of prescribed medications. The patient (or the proxy) may provide a scannable code (e.g., provided on a paper or digital receipt, on a key device, etc.) to the employee, and the scanning can trigger the secure container to unlock/open such that the employee can transfer the prescribed medications to the patient. The scanning can generate data (e.g., electronic data) to record an identify of the employee responsible for distributing the prescribed medications to the patient. Alternatively, in a different example, the patient (or the proxy) can scan the scannable code by using a scanner at the pharmacy or a scanner of the secure container, thereby to unlock the secure container and retrieve the prescribed medications. Prior to picking up the prescribed medications, the patient may receive a notice (e.g., an e-mail, a text message, etc.) indicating that the prescribed medications are ready for pick up at a specific secure container. In some cases, the code provided to the patient for medication pickup may be a portion of a reconstructable visual code, as described elsewhere herein, and the rest of the reconstructable visual code may be provided by the pharmacy or the secure container. Upon reconstruction of the reconstructable visual code, the patient may be able to pick up the prescribed medications from the secure container. In some examples, the pharmacy and/or the secure container may comprise one or more cameras for tracking and/or recording the individual picking up the prescribed medications.

Once the container with adapter and contents is transported to the home of the patient, or to a location where the patient wishes to unlock the container, the patient may use another unlock code on the key device, to unlock the container and access the medication. The key device may be similar to the key devices described elsewhere herein. The key device may be the same device that is used by the patient or caregiver to pick up the container secured with medication from the pharmacy. Alternatively, the key device used at home for unlocking the container and accessing the medication may be separate from the device used for pick-up. The key device used at home may be implemented on a mobile device or wearable device of the patient or caregiver.

The locking and unlocking of the container/adapter to gain access to the medication can generate data indicating the number of times and when the container/adapter is unlocked or locked, when the medications were secured and later accessed, etc. The data may be linked with any smart home systems within the house, that can alert the patient, the caregiver or a healthcare provider as to when the container is locked, and when the container is unlocked to gain access to its contents. The data may be used to track the patient's adherence/compliance to the medication regimen. Additionally, the data can be used to detect possible diversion or unauthorized access to the medication.

In some embodiments, the container can be a medication dispenser container. The dispenser container can be used to track a patient's adherence of medication. The dispenser container can be configured to implement the dispensing of medications in tablet/caplet/capsule format. The dispenser container may be preloaded with medication at a retail pharmacy, hospital, pill pack facility, etc. The preloaded dispenser container can be shipped or delivered to a patient, to the patient's caregiver, or to an advocate for the patient. In some embodiments, the dispenser container can be designed to accommodate or provide a single space for each tablet/capsule. The medications can be counted and placed into the dispenser container, either manually by a person or via automated robotic mechanisms, and subsequently locked in the dispenser container. Each time a patient (or the caregiver) unlocks the dispenser container with the patient's key device (or caregiver's key device) at a location (e.g. home, office, hospital, etc.), the dispenser container/adapter-key device system can send data back to a central tracking system to track the patient's adherence to the medication schedule/routine. The data may include when and where the container is unlocked and locked each time after a round of dispense. The number of times the container was unlocked and locked, the specific time instances of each event, the amount of medication withdrawn each time, and other data can be used to track the patient's adherence. Any unusual activity (e.g. possible substance abuse, diversion, theft or loss of medication), low rate of adherence, non-compliance, and the like may result in alerts or notifications being sent to the patient, the healthcare provider or any other relevant parties (e.g. the patient's caregiver, personal nurse, proxy or advocate). In any of the embodiments described herein, images or video may be taken each time the container is opened, as the medication is being administered to the patient, when the patient consumes the medication, when the patient refuses the medication, etc. The images or video may be saved in a database, and used to track the patient's adherence or compliance, monitor for diversion, misuse, or theft, etc.

Any unused medication can be locked back into the container, and returned by the patient or caregiver to an intelligent medication takeback or return system. A unique return code may be generated on the container/adapter once the unused medication is secured within the container/adapter. When the patient or caregiver activates the return system and begin the process of the return, the return code on the container/adapter can be scanned or identified by the return system. The return system can be configured to unlock and open the container/adapter, identify its contents, image and/or capture video of the container and its contents, for example as described in U.S. Provisional Application No. 62/830,215 filed Apr. 5, 2019 (titled “Systems and Methods for Drug Take-Back”).

In some embodiments, when unused or wasted medications are returned to a take-back or return system, one or more unique codes on the container/adapter can be triggered by a physical action occurring on the container/adapter. The physical action may be manually performed by a user (e.g. nurse), or electronically activated (e.g. via wireless signals, voice commands, etc.). The physical action may include movement of a mechanism to change or activate a code. The mechanism may include a sliding, rotational, flipping, or depressing mechanism. The mechanism may include any type of movable structural elements that when moved/actuated causes a unique code to be displayed on the container/adapter. The code may include any of the codes described elsewhere herein. In some instances, the code may include one or more color codes. As an example, different color codes can be used to indicate the types of unused or wasted medications, etc.

When the container is placed into the return system, the return system can be configured to identify the contents, quantity, patient's identity (which may depend if the patient chose to be identified or to remain anonymous), time of return, date of return etc.

Data about the return and the above information may be linked to the patient's profile or sent to data center(s) that monitor utilization or consumption of medication. After the data has been collected, the container and its contents may be destroyed or scheduled for waste. In some embodiments, only the contents may be destroyed, and the container may be reused by the pharmacy for other patients.

The medications that can be secured and transported using any of the containers with adapters described herein may include prescription medications (e.g., controlled substances) or non-prescription drugs. The medications may be pharmaceuticals or nutraceuticals. The medications may include any other therapeutic treatment modalities using passive or active treatment methodologies. The medications may be any other compounds (natural, synthetic, modified natural, or combinations thereof). Any type of oral medication (ingested, inhaled, etc.) may be used with the containers described herein.

Various components of the system (e.g., container, adapter and/or key device) disclosed herein can include or be fabricated from materials such as polyvinyl chloride, polyvinylidene chloride, low density polyethylene, linear low density polyethylene, polyisobutene, poly(ethylene-vinylacetate) copolymer, lightweight aluminum foil and combinations thereof, stainless steel alloys, commercially pure titanium, titanium alloys, silver alloys, copper alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, glass, and combinations thereof.

Various components of the system (e.g., container, adapter and/or key device) disclosed herein may have material composites, including one or more of the above materials, to achieve various desired characteristics such as strength, rigidity, elasticity, compliance, and/or durability. The components of the container, adapter and/or key device, individually or collectively, may also be fabricated from a heterogeneous material such as a combination of two or more of the above-described materials. The components of the container, adapter and/or key device may be monolithically formed or integrally connected.

An automated dispensing machine (ADM) as described herein may be a commercially available ADM including, for example the McLaughlin dispensing system, the Baxter ATC-212 dispensing system, Omnicell, and the Pyxis MedStation. In some embodiments, one or more of the containers, adapters or key devices disclosed herein can be stored in a drawer of the ADM (e.g., a CUBIE pocket in the Pyxis MedStation).

A container as described herein can include a housing. A housing can include a casing, enclosure, shell, box, and the like. A housing can include one or more hollow chambers, cavities or recesses. The housing may be formed having any shape and/or size. The housing can be configured to support and store contents (e.g. oral medications) as described elsewhere herein.

A code as described herein may be a visual code. The term “visual code,” as used herein, can refer to optical, machine-readable, representation (e.g., marking) of data, where the data usually describes something about the article(s) carrying the visual code. In some cases, the article(s) may comprise one or more devices (e.g. the container, adapter and/or key device). The visual code can comprise one or more graphical visual elements (e.g., one or more pictorial and/or textual datagrams), including, but are not limited to, one-dimensional (1D) visual codes representing the data by varying the width or spacing of parallel lines, two-dimensional (2D) visual codes which represents the data as a geometrical pattern, such as Quick Response (QR) codes, and/or three-dimensional (3D) visual codes. In some cases, the 3D visual codes may be a layer comprising a plurality of 1D and/or 2D visual codes, or a plurality of 1D and/or 2D visual codes that are at different depths with respective to one another. At least a portion of the visual code may can be visible by the naked eye. Alternatively, at least a portion of the visual code may not and need not to be visible by the naked eye.

In some cases, the visual code can be read by a visual scanning system (e.g., a sensor), such that the visual scanning system can extract information (e.g., information about the article(s) carrying the visual code) stored in the visual code. In some cases, the visual code can be read by the visual scanning system, such that the visual scanning system can be operatively connected to an external database that contains such information. In some cases, the visual code can be read by a user.

In some embodiments, a code as described herein may be a non-visual code. For example, the code may include encrypted signals, locking/unlocking codes, electronic keys, cipher keys, authentication codes, etc.

In some embodiments, the container and/or adapter may be configured to display different colors to indicate that the securing action of the contents has been completed. The colors may be displayed on anywhere on the adapter and/or the container. In some cases, different images may be displayed to reflect that the securing/locking action has been completed.

In some embodiments, a take-back system can determine the contents of a container by unlocking the cap/adapter via a mechanism adapted for such purpose. The container can be emptied of its contents by turning the container, shaking the container or lining up the contents in a specific manner. This can enable the contents to be identified, for example the name of the item (medication), strength of the item (medication), manufacturer, color, lot #, expiration date, amount of the contents being returned, amount originally dispensed, date on which the medication was originally dispensed, date of return, name of the patient, name of the caregiver, etc.

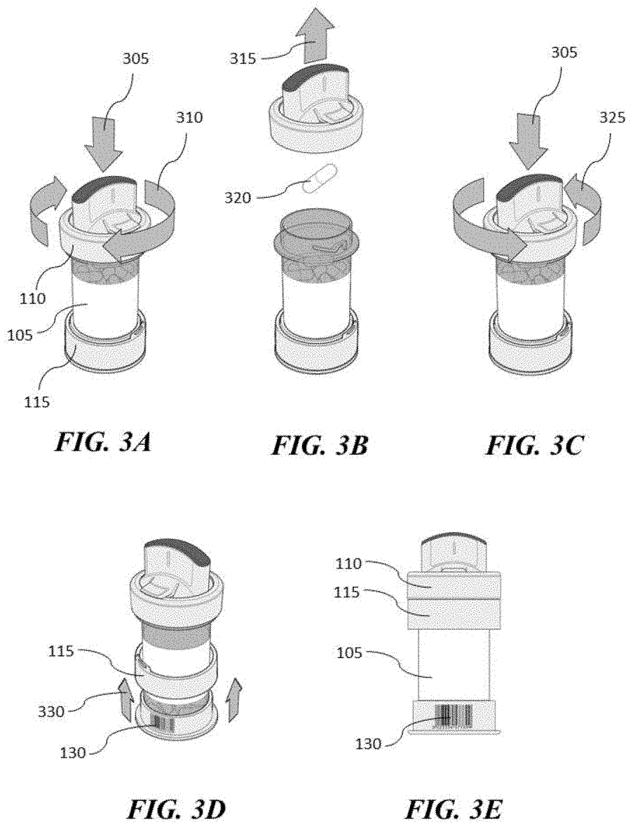

FIGS. 3A-3E schematically show methods of unlocking and securing of the oral medication container 105 using the adapter 110. Referring to FIG. 3A, the cap can be depressed (e.g., along a direction as indicated by the arrow 305) and/or twisted (e.g., along a direction as indicated by the arrow 310) to open the container and gain access to the content(s) (e.g., pill medications) inside the container. Referring to FIG. 3B, the cap can be detached form the container (e.g., along a direction as indicated by the arrow 315) to remove the content(s) 320 from the container. Referring to FIG. 3C, the cap can be depressed (e.g., along the direction 305) and/or twisted (e.g., along a direction as indicated by the arrow 325) to close the container. The directions 310 and 325 may be opposite to each other (e.g., clockwise and counter-clockwise directions). Referring to FIG. 3D, the collar 115 can be raised from the bottom of the container and towards the cap (e.g., along a direction as indicated by the arrow(s) 330). The collar can be coupled to the cap (or alternatively to a top portion of the container) to lock the cap and lock-out any unused contents (e.g., medications) inside the container. Upon movement of the collar, a unique scannable code 130 can be revealed. The unique code can be displayed on a surface of the container as shown in FIG. 3D. The locked container, as shown in FIG. 3E, can be returned to a take-back system as disclosed herein for collection and/or destruction of any unused or leftover medications.

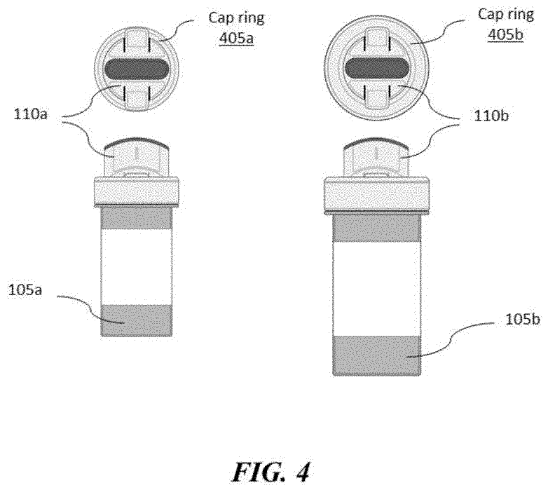

FIG. 4 schematically illustrates another example of the container 105 that is coupled to the adapter 110. The adapter can be designed with a cap ring having varied dimensions (e.g., width, circumference, etc.) to accommodate coupling to containers of varying sizes. For example, an adapter 110a can have a cap ring 405a to couple to a container 105a. An adapter 110b can have a cap ring 405b that is larger than the cap ring 405a, such that the adapter 110b can couple to a container 105b that is larger than the container 105a. In some embodiments, the adapter can be configured to track and log the time when the cap is moved relative to the container, e.g., to open and/or close the container. For example, the adapter can be operatively coupled to or comprise a controller configured to log (e.g., generate an electronic data) indicative of the time and/or the direction of movement of the adapter relative to the container. In some cases, prescription of the medications can be programmed in to the controller that is operatively coupled to or a part of the adapter.

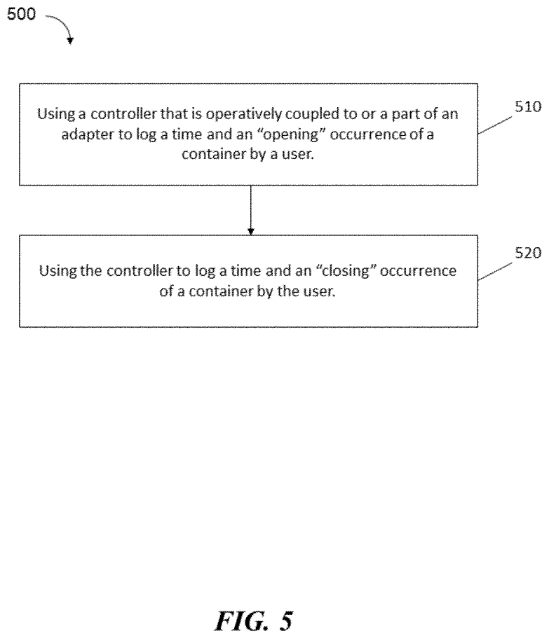

FIG. 5 shows an exemplary flowchart 500 of a method for unlocking and securing of the oral medication container using the adapter of FIG. 4. A container that is locked by an adapter can be provided to a user (e.g., a patient). The user can depress (e.g., downwards) and twist (e.g., clockwise) the adapter to open the container. The method can comprise using a controller that is operatively coupled to or a part of the adapter to log a time and an “opening” occurrence of the container by the user (process 510). The user may remove a prescribed amount of medications from the container. Subsequently, the user can depress (e.g., downwards) and twist (e.g., counter-clockwise) the adapter to close the container. The method can further comprise using the controller to log a time and a “closing” occurrence of the container (process 520). Subsequently, the user can carry the container that is locked by the adapter to a take-back system for refill of the medications. For refill, the take-back system and/or back-end user of the take-back system (e.g., a physician) can receive and analyze information (e.g., digital data) indicative of the time(s) and opening/closing occurrence(s) of the container to review usage (e.g., administration or consumption) of the medications inside the container. When such occurrences align with the prescription, a refill of the medications can be issued. Such information can be used to help the physician better manage the patient's prescription. When such occurrences do not align with the prescription, an alert can be generated by the physician or the take-back system, such that the patient can be questioned or probed for a possible medication negligence, misuse, or diversion. For example, the take-back system can be configured to determine an amount of left-over medications inside the returned container. By analyzing the information logged by the adapter and the take-back system, the take-back system and/or the physician can determine whether too much or too little medication is removed from the container with respect to the number of times the container has been opened and closed. When the take-back system and/or the physician determines that too much of the prescribed medication is removed from the container with respect to the number of times the container has been opened and closed, a diversion alert can be generated, and the patient can be required to discuss with the physician or another medication regulator (e.g., pharmacist) to further assess the possibility of illicit medication diversion.

B. Prescription Box

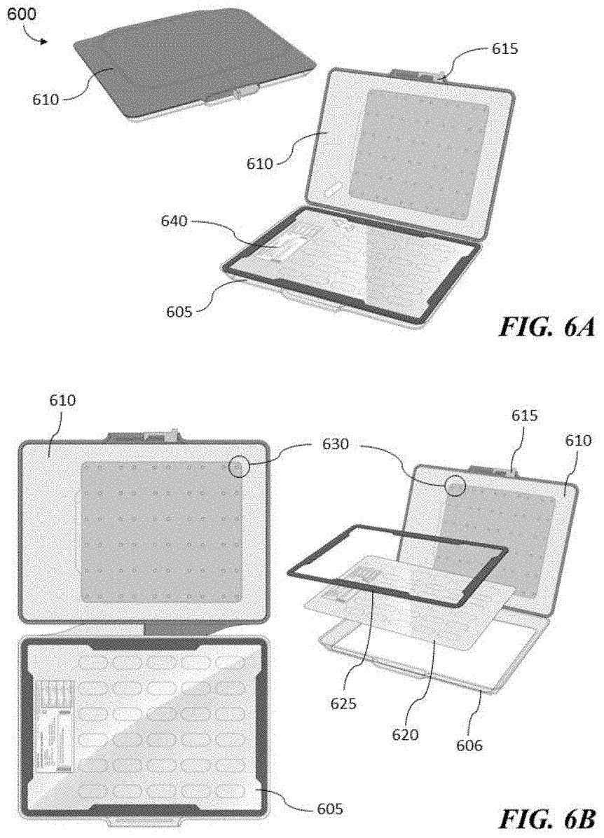

In some embodiments, the container and the adapter (e.g., as described in Section II, Part A of the Specification) can be implemented as a prescription programmed box, as illustrated in FIGS. 6A and 6B. Referring to FIG. 6A, the prescription box 600 can comprise a container 605 configured to contain the medications (e.g., oral medications, such as pills) and an adapter 610. The container and the adapter can be coupled to one another, such that a movement of the adapter relative to the container can open and/or close the prescription box. In an example, the adapter 610 can be configured to operate as a lid for the container. The prescription box can comprise a locking mechanism 615 to lock and/or unlock the coupling of the container and the adapter, such as to control access to the medications within the container. The medications can be sealed within the container of the prescription box. For example, the container can use a similar mechanism as to a blister package to individually or collectively seal one or multiple oral medications. In such a case, the oral medication(s) can be accessed only by breaking the blister seal, As described herein, the prescription box can be programmed to track use and removal of any medications from the container and generate data indicative of such usage and removal. The prescription box can be returned to a take-back system as described herein, and any data generated by the prescription box can be transferred or read by the take-back system.

Referring to FIG. 6B, the prescription programmed box 600 comprises the container 605 and the adapter 610. The adapter can be a lid to close or open the container. The container can comprise a housing 606. The housing (e.g., a guard) can be configured to contain or couple to a blister package 620 for individually sealing and containing oral medications. The container can comprise a frame 625 configured to couple the blister package to the housing. The adapter can comprise one or more sensors configured to detect presence of absence of the oral medications within the blister package. The adapter can comprise at least or up to about 1, 2, 3, 4, 5, 6, 7, 8, 9, 10, 15, 20, 30, 40, 50, 60, 70, 80, 90, or 100 sensors. For example, the adapter can comprise at least one sensor for each oral medication contained within the blister package. A sensor of the adapter can be disposed opposite of an oral medication of the blister package, such that the sensor can detect when the blister is broken, indicating release of the oral medication. One or more sensors (e.g., 1, 2, 3, or 4 sensors) can be programed to detect retrieval of each oral medication from the blister package.

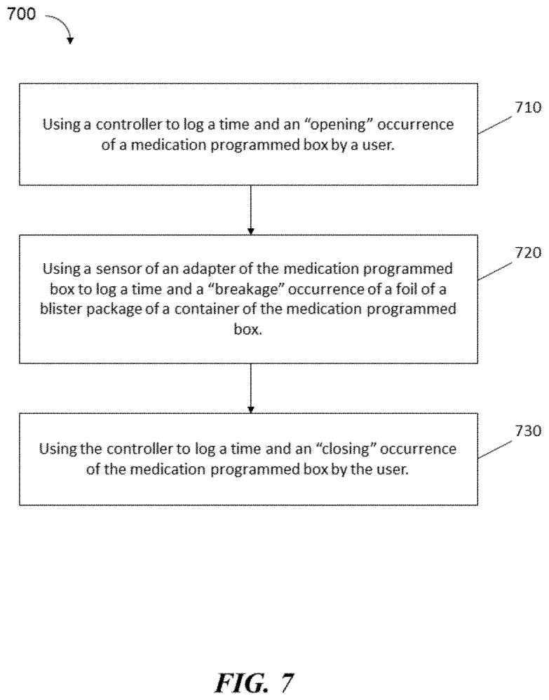

FIG. 7 shows an exemplary flowchart 700 of a method for using the prescription programmed box as illustrated in FIGS. 6A-6B. A user (e.g., a patient) can open the prescription box. The prescription box can be opened by moving the adapter (or lid) relative to the container. The method can comprise using a controller that is operatively coupled to the container, the adapter, and/or the locking mechanism of the prescription box to log a time and an “opening” occurrence of the prescription box (process 710). The user can break the blister package (e.g., break the foil of the blister package) to retrieve a pill from the blister package. The method can comprise using a sensor disposed on the adapter and against the foil for sealing the pill to log a time and a “breakage” of a position of the blister package, which log is indicative of a removal of a pill from the blister package (process 720). The sensor can be operatively coupled to the controller described herein. The user can remove a prescribed amount of medications from the blister package. The user can close the prescription box and lock the prescription box using the locking mechanism. The method can further comprise using the controller to log a time and a “closing” occurrence of the medication box (process 730). The method can comprise returning the medication box that is locked by locking mechanism to a take-back system for refill of the medications, as described herein. In some cases, the medication programmed box can be reusable by replacing a used blister package with a new blister package. Alternatively, the medication programmed box can be configured for a single use only. In some cases, the controller of the medication box can be configured to log and generate electronic data indicative of a time and occurrence of when the medication box has been opened for longer than a predetermined period of time (e.g., at least or up to 1, 2, 3, 4, 5, 10, 15, 20, 30 minutes), which can be indicative of a potential diversion of the medications. In some cases, the controller can be configured to log and generate electronic data indicative of a time and occurrence of when a plurality of blisters of the blister package is broken at the same time or within a short time period (e.g., within 1, 2, 3, 4, or 5 minutes), which can be indicative of a potential diversion of the medications. In some cases, the controller can be configured to determine how much of the prescribed medication is removed at once by analyzing a number of blisters broken at each time.

In some embodiments, the prescription box can comprise a prescription label (or any other patient and/or medication identifier) affixed thereto. For example, the prescription label can be a unique scannable code 640, as illustrated in FIG. 6A.

In some embodiments, the container and/or the adapter can be configured to be opened by a take-back system, as described elsewhere herein. In some cases, the take-back system can comprise a key that is configured to wirelessly communicate with the container and/or the adapter, thereby to instruct the container and/or the adapter to open. In some cases, the take-back system can comprise a mechanical key that is configured to couple to at least a portion of the container and/or the adapter to open the container. The container and/or the adapter can be configured to distinguish between a key device of a patient (e.g., the key device 150 as shown in FIG. 1) and a key device of the take-back system by, for example, receiving a wireless signature that is specific to each key device. For example, the container can have a separate opening (e.g., apart from the opening that is controlled by the adapter) that is specifically designed to be opened by the take-back system. In another example, the take-back system can be configured to direct movement of the adapter to open the container. Once unlocked by the take-back system, the container and/or the take-back system can be configured to generate a code (e.g., a digital data) that indicates that the container has been opened. The code can encode a time and location of the container when the container was opened. The code can comprise or digitally linked to one or more images or videos of (i) the container, (ii) the contents carried within the container, and/or (iii) the take-back system. The images or videos of the contents of the container can be captured by a sensor (e.g., a camera) of the take-back system while the contents are still within the container. Alternatively, the take-back system can be configured to remove (e.g., pour out) the contents from the container to a platform of the take-back system to capture the images or videos of the contents by using the sensor.

The container and/or the adapter as described herein can be recycled. In some cases, the container can be mechanically broken down (e.g., by the take-back system or by another instrument outside of the take-back system) in an environmentally friendly manner for recycling.

In some embodiments, the container as described herein can comprise a unique code (e.g., a unique scannable code such as a machine readable code, as described elsewhere herein). The unique code of the container can be provided on the container during manufacturing of the container. Alternatively, the unique code can be provided on the container post-manufacturing of the container, e.g., by a healthcare provider (e.g., a nurse) at a medical facility. Such unique code can be located anywhere on the container, e.g., on an outer surface, inner surface, side surface, bottom surface, flat surface, curved surface, multiple surfaces, etc.

Upon returning, the take-back system described herein can be configured to determine whether the container has been previously opened or not. In some cases, the take-back system can retrieve electronic data from the adapter of the container (e.g., via wireless communications, such as RFID or Wi-Fi), and the electronic data can comprise information as to the number of times (e.g., 0, 1, 2, 3, 4, 5, or more times) the adapter has been accessed to open the container. Alternatively, the container and the adapter can comprise a marker (e.g., a security tape) indicative of whether the adapter has been moved relative to the container to open the container. As such, when a container is returned with one or more unused medications (e.g., unused oral medications), the take-back system can be configured to distinguish between (i) unused medications in an unopened medication container and (ii) unused and leftover medications in a previously opened medication container. In some cases, the unused medications in an unopened medication container can be reused, e.g., by the patient or another patient.

### III. Carrier

The devices described herein can utilize or comprise a carrier and a key device. The carrier can be configured to carry medications (e.g., oral medications, injectable medications, etc.) or other substances (e.g. high value pharmaceutical compounds or formulations in clinical phase trials). The carrier can utilize one or more aspects (e.g., shape, design, size, means of operation, etc.) of the container and adapter for medication containers, as described in Section II, Parts A and B of the Specification. For example, the carrier may be formed having any shape, design, and/or size. The shape, design, and/or size of the carrier may be customized depending on the contents to be transported and secured (e.g. medications, medical devices, vials, syringes, needles, suture packs, etc.). The shape, design, and/or size may be ergonomically designed such that the carrier can be easily carried by a healthcare practitioner (e.g. a nurse), or easily loaded onto and removed from a shelf, docking station, rack or trolley used for holding or transporting multiple carriers. A carrier may have any regular shape or irregular shape. A carrier as described herein can be configured to carry a single type of content (e.g., a single type of medication). Alternatively, a carrier may be configured to carry a plurality of types of contents (e.g., a plurality of types of medications). The plurality of types of medications can be for a single patient or for different patients. The key device can utilize one or more aspects (e.g., shape, design, size, means of operation, etc.) as described herein (e.g., as described in Section II, Part A of the Specification).

The carrier can include a hollow interior for storing contents (e.g. medications) during transport. The carrier can include an opening for receiving contents. Access to the interior of the carrier can be regulated using a lid (or door) at the opening. The carrier can be configured to switch between an unlocked state and a locked state, by unlocking or locking the lid. Access to the interior of the carrier can be achieved by manipulating the lid, for example by moving the lid up or down in a sliding motion, flipping the lid open, or by opening a front, rear, top, bottom, or side portion of the carrier. The carrier may include a locking mechanism (e.g. an electromechanical lock) for locking and unlocking the lid. A carrier can be unlocked using a key device. A carrier and a key device may be paired with each other to form a secure transport and materials handling system. In some cases, multiple devices may be paired with one another, in a one-to-one configuration, a many-to-one configuration, or a many-to-many configuration. For example, in some embodiments, two or more carriers (containing medications and/or medical equipment for a specific patient) may be paired with a key device that is unique to and associated with that specific patient. The carrier can be configured or used to transport medication from a dispenser (e.g. an automated dispensing machine (ADM)) to a patient. The carrier is secured and locked during the transport of the medication from multiple points within a healthcare institution. Examples of such multiple points may include a point A (e.g. at the ADM) to a point B (e.g. patient's location). The carrier can be unlocked or locked any number of times, and the locking and unlocking may be location dependent. For example, the carrier can be locked after it is secured with new medication, or unused medication post administration. The carrier can be unlocked when the medication is about to be administered to the patient. The carrier can also be locked during transportation of any unused or wasted medication, and can be unlocked at the return or take-back system when the unused medication is returned for wasting/destruction. The locking and unlocking of the carrier, at different instances in time corresponding to different physical locations and different events, may trigger different codes indicating which action is being performed at the corresponding juncture. Accordingly, the codes can be used to track the dispense, transport and handling of medication, as well as the return of unused medication, in a closed loop real-time secure tracking process. The carrier is designed to be tamper-proof. Any unauthorized attempts to tamper, break into, open or hack the carrier during the secure transport process can result in notifications and/or alerts being sent, informing the relevant authorities that the carrier has been compromised. In some embodiments, the carrier can be utilized to transport expired medications and/or unopened medications in their original packaging (or their original containers).

The key device can be provided to the patient when the patient checks in or is admitted to the healthcare institution. The key device may be provided in various configurations or forms, for example as a device attached to a bedside in the patient's hospital room, as a wearable that is worn on the patient's body (e.g. wrist or other extremity), etc. In some embodiments, the key device may be provided as a software/app on a wearable device (e.g. smartwatch) or mobile device (e.g. smartphone). The key device and the carrier are paired with each other, such that a specific assigned key device is used to unlock the specific assigned carrier for a designated patient. The key device can be configured to wirelessly communicate with the carrier and unlock the carrier, when both are brought into proximity with each other near the patient (e.g. via Bluetooth, Near Field Communication (NFC)), or when the carrier is remotely unlocked (e.g. via a telecommunications network or via a secure connection over the Internet) by a key device located with a proxy or authorized advocate for the patient. Accordingly, the carrier can only be opened (by a healthcare practitioner responsible for administering the medication to the patient) when the carrier is placed near the assigned patient's key device, or when the carrier is remotely unlocked by a key device located with a proxy or advocate for the patient. This can significantly reduce or eliminate the risk of diversion as the medication is transported from a dispensing room to the patient.

A. Carrier for Inpatients

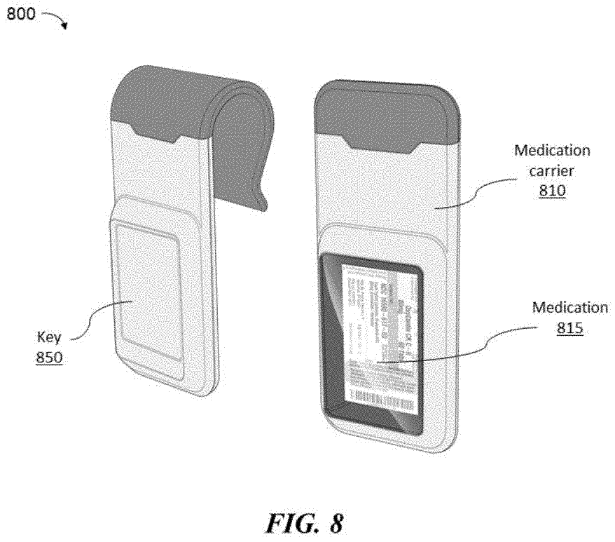

Referring to FIG. 8, a secure transport system 800 may comprise at least one carrier 810 and at least one key device 850 that is operatively coupled to the at least one carrier 810. The system can be for an institutional environment, e.g., for medications prescribed to an inpatient. The system is a dual-component system for multiple parties, namely the healthcare practitioner (e.g. a nurse) and the patient. The first component is the carrier which is generally centric to the healthcare practitioner (e.g. a nurse). The second component may be one or more key devices which are centric to the patient, and/or centric to a person who is responsible for making certain healthcare decisions on behalf of the patient.

In some cases, a patient may be unable to communicate with a healthcare provider (for example, if the patient is in a coma, physically or mentally incapacitated, has undergone a stroke, is under the effects of medication, unable to speak or write, suffering from dementia or some form of mental disorder, etc.). In those cases, a key device may be provided to a proxy or an authorized advocate (e.g. a personal nurse, a family member, or a trusted friend/associate) for the patient. Accordingly, in those cases, the key device may be centric to the proxy or authorized advocate who may be responsible for making certain healthcare decisions, and acting on behalf of and for the patient. The key device may be provided as a discrete stand-alone device to the advocate. In some embodiments, the key device may be provided on a mobile device (e.g. smartphone) or a wearable device (e.g. smartphone) of the advocate's. In some cases, the advocate for the patient may be at home or on the road, and can receive a notification from the system that the nurse is at the patient's bedside (as verified by proximity to a key device on the patient's wristband or bedside). If the patient is unable to communicate with the nurse and the advocate has prior authorization to make certain medical decisions on the patient's behalf, the advocate can use his/her key device to unlock the carrier remotely and allow the nurse to dispense the medication.

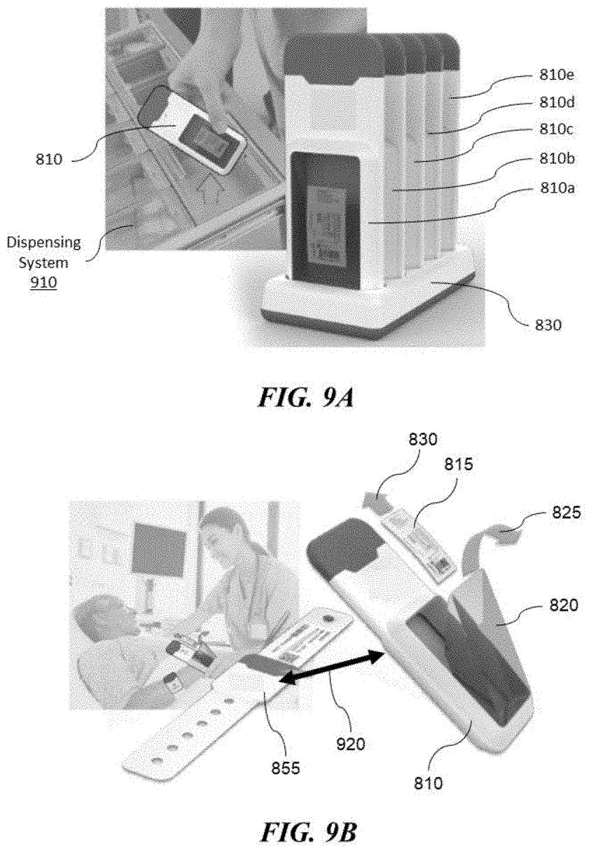

FIGS. 9A and 9B show examples of a carrier paired with a key device. FIG. 9A shows scheduled medications being retrieved through a computerized ordering and dispensing system 910. The medications can be securely stored in the carriers 810 described herein. In some cases, the dispensing system (e.g., ADM) can comprise a docking station 830, and the docking station can be configured to hold a plurality of carriers 810a, 810b, 810c, 810c, 810d, and 810e. In some cases, one or more of the carriers 810a-e can be operated based on electric energy (e.g., via a battery), and the docking station can be configured to charge one or more of the carriers 810a-e. The carriers can be used to transport the medications in a secure manner to the patient. A carrier as described herein may be a transport sleeve or pouch. Referring to FIG. 9B, a carrier 810 comprising the medications 815 can communicate (e.g., wirelessly as indicated by the arrow 920) with a key device 855 located with the patient or a key device located with the patient's advocate. The communication may be wireless or wired, and can be enabled using one or more radio communication chips. Wireless communications may include Bluetooth, near-field communication (NFC), Wi-Fi, Wi-LAN, radio-frequency identification (RFID), infrared (IR), etc. Upon pairing of the key device and the carrier, the carrier can be unlocked when it is brought in proximity to the key device, a lid 820 of the carrier can be opened (e.g., along a direction as indicated by the arrow 825), and its contents (e.g., medications 815) accessible (e.g., manually removed along a direction as indicated by the arrow 830) for bedside administration to the patient. In some cases, the carrier can be unlocked by a key device located with a proxy or advocate for the patient. In some instances, the carrier can be remotely unlocked by the advocate's key device if the advocate is not physically present with the patient. In certain cases, the carrier may be unlocked using only the patient's key device. In other cases, the carrier can be unlocked using both the patient's key device and the advocate's key device in combination as a dual-unlocking device. In some embodiments, the key device may be hung from the patient's bed using the U-shaped hook as shown in FIGS. 1 and 4. In other embodiments, the key device may be provided as a wearable (e.g. a wrist-strap) that is worn on the patient, for example as shown in FIG. 9B. The key device may be located anywhere, for example on the patient (e.g. a Patient Identity (ID) Bracelet), near the patient, patient's mobile device (e.g. smartphone) or wearable device (smartwatch), patient's bed, etc. In some embodiments, the key device may be located remote to the patient, for example with a proxy or an authorized advocate for the patient. As previously described, the key device for the advocate may be provided on a mobile device and/or wearable device of the advocate. The key device may be provided in the form of an app loaded on the mobile device or wearable device. The key device may require the advocate to enter a password to unlock the carrier. Alternatively, the key device may require the advocate's biometric identification (e.g. fingerprints, iris scans, facial recognition scans, etc.) to unlock the carrier. In some cases, a code may be sent to the advocate's mobile device or wearable device, and the advocate has to enter the code into the app to unlock the carrier.

The key device(s) and the carrier(s) may be dispensed from automated dispensing machines (ADMs), from central pharmacies, from a hospital's central supply, or from any suitable location that is accessible by healthcare practitioners and patients. In some embodiments, the carrier(s) may be dispensed outside of the ADM. For example, the carriers may be located and stored on a carrier shelf. The carrier shelf may include a plurality of dispensing ports/drawers for storing a plurality of carriers. The dispensing ports/drawers may be used to store carriers of different shapes and/or sizes. The carriers may be locked within the ports/drawers, and accessible upon request (e.g. entering a code for one or more types of carriers). When a specific carrier is being requested, the port/drawer carrying that specific carrier may extend out of the shelf, thus allowing a nurse to access and retrieve the carrier. The carrier may be initially in an unfilled state, such that the carrier can be filled with a medication retrieved from an ADM (and to secure the medication within). In some alternative embodiments, the carrier may be initially in a filled state, whereby the carrier may be provided with the medication locked within. As an example, personnel at a pharmacy may load one or more unfilled (or filled) carriers into Pyxis, other ADMs, or stored areas in a medication room. In some embodiments, the key device as described herein can be dispensed from an automated machine (e.g., an ADS in a medical facility), a vending machine at a facility, or an individual (e.g., a supervisory personnel) responsible for distribution of the key device. Depending on the contents, the automated machine can be disposed within a medical facility or a non-medical facility (e.g., a food processing factory, a medication manufacturing factory, a military base, an evidence storage room of a police station, etc.).

In some embodiments, a carrier can be used to transport contents (e.g. medications, medical devices, hospital supplies, etc.) from one healthcare provider/institution to another healthcare provider/institution. As an example, the carrier may be provided separately from a central pharmacy. The medication or medical supplies may be provided separately from a dispensing facility. The carrier and the medication or medical supplies can be subsequently provided to the room where the patient is located. Prior to administering the medication or medical supplies, the key device at the patient's bedside (or a key device located with the patient's advocate, either within the hospital room or remote to the hospital) is required to unlock the carrier containing the medication or medical supplies. In some embodiments, the carrier can be used to transport (or dispense) narcotics from a central pharmacy to an institutional destination (e.g., a particular floor or a room of a hospital, nursing homes, etc.).

In some embodiments, a carrier can be used to transport borrowed or loaned contents (e.g. medical devices, hospital supplies, medications, etc.) between healthcare providers/institutions. For example, a carrier can be used to securely transport any of the aforementioned contents from one healthcare provider/institution to another healthcare provider/institution. In some instances, some of the contents (e.g. medical devices or equipment) may be loaned and/or borrowed, and may be returned to the originating healthcare provider/institution after completion of use. Examples may include any reusable medical devices or equipment. In some cases, a carrier can be used to carry contents from a first healthcare provider/institution for replenishment at a second healthcare provider/institution. When the contents have been replenished at the second healthcare provider/institution, the second healthcare provider/institution can use the carrier to return the contents back to the first healthcare provider/institution.

When a medication is ordered for a patient, the ADM can dispense the medication from the proper drawer or pocket using standard retrieval logins and methods, in accordance with the policies and procedures for that particular institution. After the medication is dispensed from the ADM, another drawer or pocket can dispense a carrier. The dispensing of the medication and the carrier may be concurrent (e.g. a one-step process). In some cases, the dispensing of the medication and the carrier need not be simultaneous. For example, the dispensing of the medication and the carrier may be performed in two or more steps using a multi-step process.

In one embodiment, the medication and the carrier may be provided separately. As previously described, in some embodiments, the carrier(s) may be dispensed at or outside of the ADM. For example, the carriers may be located and stored on a carrier shelf. The carrier shelf may include a plurality of dispensing ports/drawers for storing a plurality of carriers. The dispensing ports/drawers may be used to store carriers of different shapes and/or sizes. The carriers may be locked within the ports/drawers, and accessible upon request (e.g. entering a code for one or more types of carriers). When a specific carrier is being requested, the port/drawer carrying that specific carrier may extend out of the shelf, thus allowing a nurse to access the carrier. The carrier may be initially in an unlocked state. Next, the nurse may insert or place the medication into the carrier, and then scan the carrier using a scanner. This may lock the medication within the carrier, and also remotely link the carrier to an assigned patient's key device.

Each carrier and each key device may be provided with its own unique code (or identifier), as described herein. The code on an assigned key device can be designed to match or pair with the code on the designated carrier, in order to unlock and open a carrier to retrieve its contents (medications and/or medical devices).

A healthcare practitioner (e.g. a nurse) at the ADM may scan the code on the carrier using a scanner, which would electronically link information about a specific medication and that specific carrier to a designated patient. At the same time, the scanning of the code on the carrier can also link the carrier with a key device associated with the designated patient. The scanning of the code can also link to the prescription order for the designated patient. The linking can be used to track the name of the medication, dosage, name of the nurse retrieving the medication, name of the patient, and time of retrieval of the medication. Only medications for the designated patient which has a valid prescription order from a physician (or other prescriber) can be retrieved from the ADM. The retrieval of a medication other than the one ordered for that patient, or an incorrect dose of the medication ordered for that patient, can trigger a discrepancy signal and a report to management of the healthcare institution, signifying an improper medication retrieval from the ADM.

Next, the medication can be placed into the empty carrier by the nurse and locked. This can occur on site or near to the ADM, in a medication room, or elsewhere in accordance with policy and procedure. Once the carrier is locked, the system can generate a secondary code, which would be unique to that specific medication and patient, with a timestamp indicating that a locked medication is now contained within the carrier. The secondary code may include or utilize any of the codes described elsewhere herein. The nurse may scan the code which may subsequently send an alert/signal to a central tracking system (and/or EMR, Pyxis, etc.) that signifies the carrier has been secured with the corresponding contents, and that the carrier is now en route to the patient, or in some instances to another healthcare provider/institution. In some cases, the carrier with its contents may be utilized at one healthcare provider/institution followed by a next healthcare provider/institution, and so forth. Accordingly, the carrier with its contents may be unlocked, unloaded and used at multiple different locations and at different points in time.

If there are additional medications for that patient, the nurse may proceed to retrieve those medications from the ADM. Once all of the medications have been retrieved from the ADM (or other systems from which the nurse typically obtains medications), the nurse may choose to place the appropriate medications into a single carrier, or all of the medications individually into separate carriers. For example, the nurse may then load and secure those medications either within different pockets/chambers in a same carrier, or load and secure each medication in a different carrier. Accordingly, in some embodiments, a carrier can be used to hold and transport only one medication for a patient. In other embodiments, a carrier may include multiple pockets/chambers for holding and transporting different medications for that patient. If multiple carriers are used, the process for loading and locking each individual carrier may proceed in a similar manner as described above.

In some embodiments, a docking station 830 may be provided for holding a plurality of locked carriers 810a-e with their contents, for example as shown in FIG. 9A. The contents may include medications, medical devices or equipment, blood, stock medical supplies such as creams, lotions, Purified Protein Derivative (PPD) solutions, IV fluids, among others. In other embodiments, a docking station may be used to hold carriers with or without contents. In some cases, a docking station may hold one or more carriers that have contents locked within, and may also hold one or more unlocked empty carriers. Accordingly, a docking station can be used for holding any carriers regardless of their state (loaded or unloaded), and capable of holding multiple carriers of a variety of different shapes and/or sizes. The docking station can serve as a communication intermediary. The docking station can be configured to assign specific carriers to specific patient-centric key devices. The docking station can also be configured to track an amount of time that expires for delivery of medication, and/or return of unused medication as described elsewhere herein. The docking station can also be used to manage the stocking/inventory of medication. In some embodiments, the docking station can be used as a charging station for charging a power source (e.g. a battery) on the carrier. The power source can be used to actuate a switch or motor to open or lock a lid of the carrier, generate a code on a display or screen on the carrier, enable wireless communication with the key or a server or electronic health record (HER) system, power audio components (e.g. speakers) on the carrier for auditory alerts/notifications, etc. In some embodiments, the carrier may include a position sensor that tracks the location of the carrier substantially in real-time as it is being moved around within the healthcare institution.

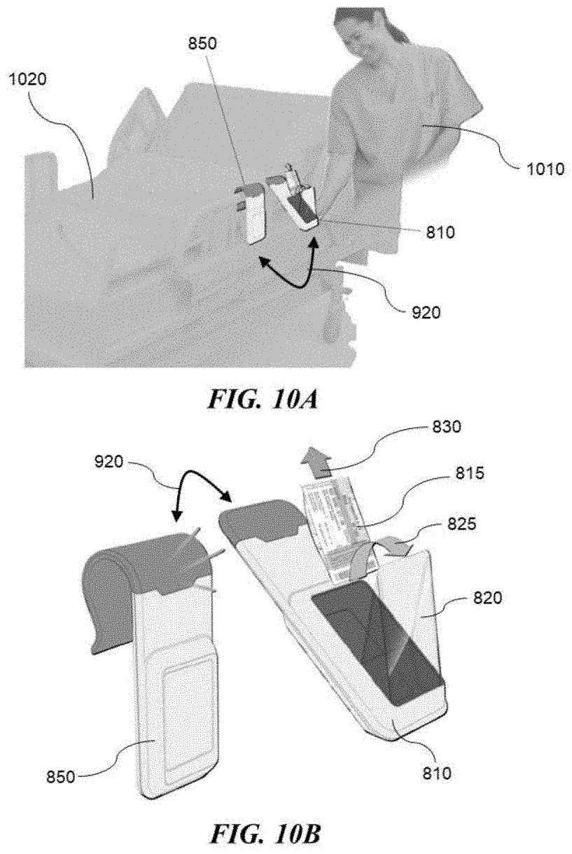

Next, referring to FIG. 10A, the nurse 1010 may bring the loaded/locked carrier(s) 810 to the patient (e.g., a patient disposed on a patient bed 1020) for administration. When the nurse arrives at the patient's bedside with the carrier, the nurse may place the carrier 810 in proximity to a key device 850 near the patient (or disposed on a part of the patient's bed 1020). In some embodiments, if the patient is non-communicative (e.g. in a coma), when the nurse arrives at the patient's bedside, an alert may be transmitted to a key device held by a proxy or advocate for the patient. The alert may inform the advocate that the nurse is about to administer the medication to the patient, and is requesting the advocate to unlock the carrier (either in-room or remotely) such that the nurse may access and administer the medication. The key device can be placed on the bedside rail as shown in FIG. 10A, or in some other location in the patient's room. The key device is specific to the patient, and is assigned to that patient when the patient is admitted into the hospital, or admitted to the floor where therapeutics are applied to the patient. As described elsewhere herein, the key device may be in the form of a Patient ID bracelet worn on the patient, or a device mounted on the bed. In some embodiments, the key device may be provided as a subcutaneous wireless device implanted into the patient's body. In some embodiments, the carrier described herein can carry food for the patient for real-time tracking of the patient's dietary consumption. Opening and/or closing of a lid of the carrier can be recorded as an indication of, for example, when the patient starts eating the food and/or when the patient finishes eating the food. In some cases, the carrier can be returned (e.g., to a carrier docket or a return area), and a picture or a video of the carrier can be taken and linked to the particular patient to analyze (e.g., compare) what was assigned to the patient and what was consumed by the patient. The patient can be at home (and treated by a caregiver), a nursing home, a hospital, a hospice, etc. Any information generated with regards to the carrier can be shared with (e.g., sent to) one or more family members of the patient.

Referring to FIG. 10B, the key device 850 can be used to unlock the carrier 810 once communications have been established, thereby allowing the nurse to retrieve the medication(s) 815 from the carrier 810. The carrier can be unlocked using signals transmitted wirelessly 920 (e.g. via Bluetooth, RFID, Near Field Communication (NFC), Wi-Fi, Infrared (IR), etc.) between the key device 850 and carrier 810. In some embodiments, the carrier and the key device may utilize a reconstructable visual code. The reconstructable visual code may be segmented, and the different visual code segments may be provided to a paired carrier and key device. For example, a first visual code segment may be displayed on the carrier, and a second visual code segment may be displayed on the key device when the carrier is brought in proximity to the key device. The nurse may place the carrier adjacent to the key device to effectively reconstruct the unique pattern of the visual code. The unique pattern may be detectable by a visual detecting device (e.g., a handheld QR reader/scanner). Upon reconstruction of the visual code, the carrier can be unlocked, thereby permitting the nurse to access the medication(s).

Referring to FIG. 10B, a lid (or door) 820 of the carrier 810 can be unlocked and/or released using the key device 850. In some embodiments, unlocking of the carrier may include causing the lid of the carrier to automatically slide or flip open. Once the lid of the carrier containing the medication is opened, another unique code may be triggered appearing on the carrier. The nurse may be required to scan the code when the code appears, and prior to administering the medication to the patient at the bedside via oral, injectable or infusion means. Scanning of the code collects information indicating when the system has been opened by the nurse, for the designated patient, for the designated drug, at that given instance in time.

In some embodiments, a return carrier containing the unused, leftover, unopened, and/or expired medication may be returned to the ADM or to a take-back system for recirculation, as described herein. In some instances, should the patient refuse the medication, or if the medication is not completely administered to the patient, the nurse may use a return carrier to store and secure/re-lock the unused medication. The return carrier containing the unused medication may be returned to the ADM or to a take-back system for recirculation, as described herein. In some embodiments, refused or returned medications in a carrier can be identified using different color codes, or other types of codes or unique identifiers that are triggered for certain events, for example when the medication is refused and/or unused medication is returned. The color codes or other unique identifiers may be triggered electronically by activating a switch on the carrier. Alternatively, the color codes or other unique identifiers may be triggered manually, for example by the user (e.g. nurse) sliding, pulling, or physically activating a mechanism which causes the specific code to appear, or which causes a change in an original color code of the carrier. As an example, a carrier used to transport medication to the patient may have a first color code (e.g. white). When the patient refuses the medication, the color code on the carrier may switch from the first color code (white) to a second color code (e.g. red). In another instance, if the medication is not completely administered and the carrier is used to return the residual medication, the color code on the carrier may switch from the first color code (white) to a third color code (e.g. blue).

The return carrier may be the same carrier that was used to transport the medication to the patient. Alternatively, the return carrier may be another new carrier that is different from the one used to transport the medication from the ADM to the patient. The return carrier can be used to return or waste the unused medication. The return carrier, regardless whether it is the same carrier used in the initial transport of the medication, or a new different carrier, has to be assigned to the designated patient, and synchronized with the timing at the patient's bedside.

The return carrier may have a unique code associated with it. The unique code can be used to link the prescription order to the return carrier. After the nurse loads and locks the unused medication in the return carrier, the nurse may scan the code using a scanner, which would then trigger or create an assignment for the waste process for that unused medication and designated patient with the locked return carrier. Once the locked return carrier has been activated, the code can be scanned into the EMR, Pyxis, or both etc., thereby linking the patient, unused medication to be wasted, and the nurse who is responsible for the patient and administration/transport of the medication. Additionally, the information can be linked to a proxy or advocate for the patient, so as to alert the advocate about the latest and relevant events (e.g. medication successfully administered, or patient refused the medication, etc.). This can provide real-time updates and timely awareness to the advocate about the patient's care/status.

In some embodiments, when the return carrier containing the unused/wasted medication is scanned, an image of the unused medication inside of the locked carrier may be captured. The image can be linked with the specific nurse, unused/wasted medication, designated patient and specific return carrier. The images and return carriers can be incorporated into an intelligent medication return or take-back system for patients in hospitals and other institutions, as described herein.

As shown in the above, the series of discrete events, starting (1) from the dispense process at the ADM or other dispensing system for a particular patient, (2) secure transport of the medication between physical locations within the healthcare institution to the patient, (3) the administration of the medication to the patient, and (4) the secure return or wasting of unused medication, is a closed loop tracking process containing various checkpoints and safeguards. This ensures a high level of security and accountability for medications and/or medical devices, which can reduce or eliminate the risk of diversion within the institution.

Each of the key device and the carrier may be disposable or non-disposable. Each of the key device and the carrier may be configured for multiple use, or for single one-time use. Each of the key device and the carrier may be made of a variety of materials, for example plastics, vinyl, cardboard etc. Unique codes for the carrier and the key device may be displayed on any portion thereof.

The carrier may include a lid that can slide or flip open (or close). The lid may include a display window or panel. In some embodiments, a unique code for the carrier (when it is locked and holding a medication within), may appear in the display window or panel. In other embodiments, the display window or panel need not be on the lid, and may be provided anywhere on the carrier, for example on a front or back surface of the carrier.

As described above, the key device may be provided at the bedside of the patient, for example coupled to the hospital bed, or other types of patient room furniture (e.g. bed stand, wheeled tray, etc). In some other embodiments, the key device may be free-floating and need not be separately coupled to the hospital bed of patient room furniture. For example, the key device can be built as an integrated component of the hospital bed, in the same manner that the nurse call button or TV remote control is built into the sides/rails of a hospital bed. This ensures that the key device is affixed at a known location, and building it into the hospital bed reduces the likelihood of tampering with the key device. In some embodiments, the key device may be part of the patient's ID wrist band, or a subcutaneous wireless device implanted in the patient. The key device may be electronic-activated, light-activated, voice or audio-activated, biometrically activated (e.g. fingerpints, eye scans, facial scans), etc. The key device can be updated with new patient information each time a new patient checks into the hospital room. As such, the key device can be dynamically configured for use with different patients over time.

Hospital beds often have power built in due to the electromechanical systems in the beds (e.g. turning motors, head up/down positioning and leg up/down motors, light/TV controls, etc.). In some embodiments, the key device may be powered using the electrical system in the bed. In some other embodiments, the key device may be powered by a single use or rechargeable battery. In some cases, the key device may be integrated as part of the bed, and configured to utilize the power and other electronic controls within the bed. The key device may be integrated into the siderails or any part of the bedframe, for example in a manner similar to the integration of nurse call buttons, TV controls, or light controls into the siderails of the hospital bed.

When the system (comprising the carrier and locking device) is activated/locked/opened/used, signals can be sent to the ADM/eMAR segment of the EHR system confirming the locking/use/opening of the carrier and the functions (e.g. retrieval, drug administering, wasting, etc.) that are being performed. In other embodiments, alerts can be sent to monitoring stations (e.g., FedEx, UPS, USPS, a military stations, a police station, a private security personnel, etc.). The alerts can be indicative of, for example, intentional (planned) opening, accidental opening, and/or unauthorized opening (or attempts thereof) of the carrier. Accordingly, the system serves as a closed loop tracking system, and can help to verify/confirm/ensure that the medication secured in the carrier is timely and appropriately delivered to the patient at the bedside. The disclosed system and its various checkpoints and safeguards can help to deter, reduce or eliminate potential diversion behavior, which is a systemic issue that current protocols (e.g. those based on an “honor” system) are unable to rectify.

The system described herein can also be utilized as an integral component of the wasting process for injectable medications. Currently, used syringes (with or without residual medication left after injection) and medication vials (with or without residual medication left after the medication is drawn up into the syringe) are wasted and disposed of in several different manners, including the use of sharps containers, squirting the excess waste medication into a sink or Cactus system, or disposal into a biohazard bag or other container. All of the above scenarios are fraught with opportunities for diversion, exposure and other risks of contamination and injury to healthcare practitioners or others.

The carrier disclosed herein can be configured to be used with injectables. For example, the carrier can include, as a fitting within the carrier, a set of clips and/or pre-sized spaces to accommodate one or more used syringes (while leaving room for a partially but not completely depressed syringe plunger). The set of clips and/or pre-sized spaces can also be configured to accommodate one or more medication vials (of a same size or different sizes). The set of clips may be a retention mechanism, e.g. snap-fits, spring-clips, flexible posts, etc. Following the administration of the injectable medication to the patient, the nurse can place the used syringe (with or without residual medication onboard) into the preset location in the carrier. In a similar manner, the nurse can place the vial from which the injected medication was drawn (with or without residual medication within) into the preset location in the carrier.

The carrier can be used with an intelligent wasting system, intelligent expired meds return system, unused/unopened intelligent medication return system, unused/unopened food intelligent return system, etc. A unique code on the secured and loaded carrier can be scanned to link the information of the waste medication to the intelligent wasting system. Additionally, images of the contents (e.g., syringe, vial) within the carrier can be captured for visual confirmation and verification. The carrier can then be placed into the appropriate section of the intelligent wasting system for destruction by a DEA reverse collector. The need for sharps containers, Cactus products or sinks can be reduced or eliminated using the above carrier and the intelligent wasting system.

In another embodiment, the carrier may include an empty reservoir for holding excess injectable medication. A healthcare practitioner may squirt or inject the excess injectable medication into the reservoir within the carrier, thereby securely storing the excess injectable medication within the carrier. This can help to prevent diversion of the residual medication. The reservoir can be designed such that it is accessible only in an inbound direction (i.e. from outside to inside of the carrier), and non-accessible in an outbound direction. Accordingly, once the excess injectable medication is injected into the reservoir, removal of the medication from the reservoir is physically restricted. Any tampering of the carrier in an unauthorized manner to extract the residual medication from the reservoir may result in notifications being sent to the relevant healthcare management authorities. In some embodiments, the reservoir may be removable from the carrier. For example, a reservoir containing excess injectable medication may be removed from the carrier for destruction. After the used reservoir has been removed, a new empty reservoir may be inserted or placed into the carrier.

It should be appreciated that any of the above described embodiments may be used as stand-alone, or can be modified and used in combination with one another. For example, in one embodiment, the carrier may include a reservoir for holding excess injectable medication, as well as a set of clips or pre-sized spaces for holding empty syringes and vials.

The individual code for each carrier and key can be reset to enable multiple, sequential uses of carriers with keys. The system can also be reset when a patient is discharged from the hospital, and when another patient requiring medication is going to use the same key device. The carriers and keys may be reset, paired, assigned, or synchronized in any combination to support usage with a large number of incoming/outgoing patients at a healthcare institution.

In some institutions, medications and other devices/equipment may be moved from one place to another using a pneumatic tube system. For example, specialty medications that are not stocked in the treatment area can be delivered from the central pharmacy to the treatment area using a pneumatic tube system. Many hospitals and other institutions with existing pneumatic tube systems have a list of “Do Not Tube” medications. These lists are typically published by pharmacy directors and pharmacy departments. The lists of prohibited medications which should not be transported via pneumatic systems are due to several reasons, some having to do with potential changes to the chemical makeup of the drug due to the pressurized status inside the tube. An increasingly important reason is the prohibition against moving controlled substances because of the increased risk of diversion, given that most existing pneumatic systems do not have security systems in place to reduce or eliminate diversion.

The systems described herein can improve the security of pneumatic systems in healthcare institutions and reduce/eliminate risk of diversion. For example, the carrier described herein can be used as a “subway-car” type transport device for secure point to point transfer and tracking of medications, medical devices, and/or other equipment from one location to another within the building. The carrier can be used with a pneumatic tube system, or any other types of conveyance system (e.g. belts, conveyors, chutes, overhead ceiling or wall rails, etc). The conveyance or transport systems may have one or more channels, for example one or more outbound channels, and one or more inbound channels. The channels may be connected to different rooms and areas within the healthcare facility. At any given instance, multiple carriers containing a variety of medications and/or medical devices can be moved through the channels for distribution to multiple patients. The carriers and key devices can enable tracking of the contents at both the sending end and the receiving end, thereby creating an unbroken chain of custody for the contents transported by the carriers. Additionally, the use of multiple tracking/scanning points within the pneumatic system (entry point, waypoints in transit, and retrieval point) using the visual code technology described elsewhere herein can help to enhance the security of the system.

In some embodiments, the carrier can be used beyond the boundaries of the healthcare institution. For example, a human courier may use the carrier to transport medications or other objects from a remote off-site location (such as pharmacy located elsewhere), on an as-needed basis, to a treatment area such as a ward or nursing station on a floor of a hospital.

In some embodiments, the carrier can be configured to hold, secure, and transport prepackaged kits containing medical instruments and devices. Examples of medical instruments and devices may include stents, valves, catheters, needles, sutures and the like.

In some embodiments, the carrier can be adapted and compatible with refrigeration environments. For example, the carrier can be loaded with medication (such as insulin) or items that require refrigeration to preserve the viability and shelf-life of those medication or items. The carrier can be configured to monitor the temperature of its contents. The carrier can be used to maintain the medication or items below room temperature. The carrier may include insulating materials for reducing heat transfer from an ambient environment into the carrier. In some other embodiments, the carrier can be configured to provide cooling/refrigeration to its contents. In some embodiments, the carrier may include a thermometer that displays an internal temperature of its chamber. In some cases, the carrier may include a temperature visual indicator that changes color with temperature. Different colors can be used to indicate different temperatures or temperature ranges. For example, a first color may be used to indicate a first temperature range, a second color may be used to indicate a second temperature range, and so forth. The colors may be provided along a gradient (for example, ranging from light blue to dark blue, light orange to dark orange to red, etc.) to indicate different temperature ranges or heat intensity.

In some embodiments, the carrier can be used to transport contents (e.g. medication and devices) from originating entities (such as pharmaceutical companies, contract manufacturing organizations (CMOs), manufacturers, wholesalers, or distributors) to healthcare institutions (hospitals, clinics, elderly care facilities), retail pharmacies, etc. Certain medications such as temperature controlled/refrigerated medications (Ativan injectable being an example) may be locked and transported in a carrier for delivery from the originating entity to the healthcare institution or retail pharmacy. The carrier can also be used to secure and transport non-refrigerated controlled medications, such as cough syrups, tablets, IVs, capsules. By locking the medications in one or more carriers prior to leaving the originating entity (wholesaler/manufacturer), the contents can be tracked. More importantly, notifications can be sent to the originating entity as to when/where the carriers were delivered and also when/where the carriers were opened and their contents retrieved. The transportation of such contents using the carriers between different entities may include the use of reconstructable visual codes as described herein, whereby segments of a code are visually reconstructed to form a full unique code in order to unlock a carrier to access its contents. Accordingly, the security systems described herein can provide a safer transportation and handling model for the pharmaceutical industry and healthcare sector as a whole.

In some embodiments, the carrier or a portion of thereof may have different color codes. The different color codes can be used to identify the classes of contents within those carriers, for example medications, unused/wasted medication, syringes, vials, or other valuable items, etc.

B. Carrier for Outpatients

The systems and methods described herein can also be applied to outpatient environments. For example, the system can be used with dispense of medication at the retail level (e.g. an outpatient pharmacy), secure transport, and access to the medication at the patient's home or other non-institutional locations.

A carrier may be initially provided at or to the pharmacy. The carrier may or may not contain the dispensed medication(s). The carrier may be activated and locked/unlocked at the time of dispensing. The carrier and device may be matched to the specific pharmacist, patient, medication, at a given point in time. The unique code generated by a locked carrier can be scanned at the retail/outpatient dispensing level, thereby linking the above information.

When the patient or caregiver is picking up the carrier containing the dispensed medication, the patient or caregiver may show a code for pickup that correlates with the locked carrier. The code may be provided on a key device held by the patient or caregiver. In some embodiments, the key device may be implemented on a mobile device or wearable device of the patient or caregiver. When the pharmacist scans the pickup code, a signal is generated indicating that the medication secured within the carrier has been picked up. The tracking of the carrier may be monitored using a combination of software and hardware at the retail drug store level, and/or at designated patient or caregiver level.

Once the carrier is transported to the home of the patient, or to a location where the patient wishes to unlock the carrier, the patient may use another unlock code on a key device, to unlock the carrier and access the medication. The key device may be similar to the key devices described elsewhere herein. The key device may be the same device that is used by the patient or caregiver to pick up the carrier with medication from the pharmacy. Alternatively, the key device used at home for unlocking the carrier and accessing the medication may be separate from the device used for pick-up. The key device used at home may be implemented on a mobile device or wearable device of the patient.

The locking and unlocking of the carrier to gain access to the medication can generate data indicating the number of times and when the carrier is unlocked or locked, when the medications were secured and later accessed, etc. The data may be linked with any smart home systems within the house, that can alert the patient or a healthcare provider as to when the carrier is locked, and when the carrier is unlocked to gain access to its contents. The data may be used to track the patient's adherence/compliance to the medication regimen. Additionally, the data can be used to detect possible diversion or unauthorized access to the medication.

In some embodiments, the carrier can be a medication dispenser carrier. The dispenser carrier can be used to track a patient's adherence of medication. The dispenser carrier can be configured to implement the dispensing of medications in tablet/capsule format. The dispenser carrier may be preloaded with medication at a retail pharmacy, hospital, pill pack facility, etc. The preloaded dispenser carrier can be shipped or delivered to a patient, or to an advocate for the patient. The dispenser carrier can be designed to accommodate or provide a single space for each tablet/capsule. The medications can be counted and placed into the dispenser carrier, either manually by a person or via automated robotic mechanisms, and subsequently locked in the dispenser carrier. Each time a patient (or an advocate for the patient) unlocks the dispenser carrier with the patient's key device (or advocate's key device) at a location (e.g. home, office, hospital, etc.), the dispenser carrier-key device system can send data back to a central tracking system to track the patient's adherence to the medication schedule/routine. The data may include when and where the carrier is unlocked and locked each time after a round of dispense. The number of times the carrier was unlocked and locked, the specific time instances of each event, the amount of medication withdrawn each time, and other data can be used to track the patient's adherence. Any unusual activity (e.g. possible substance abuse, diversion, theft or loss of medication), low rate of adherence, non-compliance, and the like may result in alerts or notifications being sent to the patient, the healthcare provider or any other relevant parties (e.g. the patient's personal nurse, proxy or advocate). In any of the embodiments described herein, images or video may be taken each time the carrier is opened, as the medication is being administered to the patient, when the used syringes are returned to the carrier, etc. The images or video may be saved in a database, and used to track the patient's adherence or compliance, monitor for diversion, misuse, or theft, etc.

Any unused medication can be locked back into the carrier, and returned by the patient or caregiver to an intelligent medication takeback or return system. A unique return code may be generated on the carrier once the unused medication is secured within the carrier. When the patient or caregiver activates the return system and begin the process of the return, the return code on the carrier can be scanned or identified by the return system. The return system can be configured to unlock and open the carrier, identify its contents, image and/or capture video of the carrier and its contents, for example as described in International Application No. PCT/US2020/026434 mentioned elsewhere herein.

In some embodiments, when unused or wasted medications are returned to a take-back or return system, one or more unique codes on the carrier can be triggered by a physical action occurring on the carrier. The physical action may be manually performed by a user (e.g. nurse), or electronically activated (e.g. via wireless signals, voice commands, etc.). The physical action may include movement of a mechanism to change or activate a code. The mechanism may include a sliding, rotational, flipping, or depressing mechanism. The mechanism may include any type of movable structural elements that when moved/actuated causes a unique code to be displayed on the carrier. The code may include any of the codes described elsewhere herein. In some instances, the code may include one or more color codes. As an example, different color codes can be used to indicate the types of unused or wasted medications, etc.

When the carrier is placed into the return system, the return system can be configured to identify the contents, quantity, patient's identity (which may depend if the patient chose to be identified or to remain anonymous), time of return, date of return etc.

Data about the return and the above information may be linked to the patient's profile or sent to data center(s) that monitor utilization or consumption of medication. After the data has been collected, the carrier and its contents may be destroyed or scheduled for waste. In some embodiments, only the contents may be destroyed, and the carrier may be reused by the pharmacy for other patients.

The medications that can be secured and transported using any of the carriers described herein may include prescription medications (e.g., controlled substances) or non-prescription drugs. The medications may be pharmaceuticals or nutraceuticals. The medications may include any other therapeutic treatment modalities using passive or active treatment methodologies. The medications may be any other compounds (natural, synthetic, modified natural, or combinations thereof). Other examples of medications that can be secured and transported using the carriers may include inhaler drugs (e.g. Albuterol, Symbicort, Advair, Spriva), creams, injectables (e.g. insulin), specialty medications (e.g. Humira, Hepatitis C medications, etc.). Any type of medication (ingested, inhaled, injected, intravenously applied, dermatically applied, implantable, etc.) may be used with the carriers described herein

In any of embodiments described herein, the carrier can be used for the secure transport of a covering. Examples of coverings are described in International Application No. PCT/US2019/042059 mentioned elsewhere herein. A covering may generally refer to an object that is to be adhered to a bodily surface of a subject, and removed after a period of time. The covering may comprise an adhesive material to connect (e.g., adhere, attach, bind) to the bodily surface of the subject. The covering may be pre-medicated (e.g., a transdermal patch comprising a drug). Alternatively, the covering need not be pre-medicated. The covering may comprise patches, pads, films, dressings, plasters, bandages, wrappers, strips, patches, gauzes, tapes, and the like that adheres to a bodily surface (e.g., healthy and/or wounded skin) of a subject. In some cases, the covering may be disposed over an additional covering that is adhered to the bodily surface (e.g., a pre-medicated patch) or an object (e.g., a needle assembly, such as an intravenous needle), which can protect the additional covering or the object (e.g., from damage, unintentional removal, etc.).

The covering may be flexible and/or stretchable. The covering may be transparent, semi-transparent, opaque, or not transparent. The thickness of the covering may be at least about 0.01 mm, 0.02 mm, 0.03 mm, 0.04 mm, 0.05 mm, 0.05 mm, 0.07 mm, 0.08 mm, 0.09 mm, 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, or more. The thickness of the covering may be at most about 5 mm, 4 mm, 3 mm, 2 mm, 1 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, 0.01 mm, or less.

Examples of the covering include BAND AID®, TEGADERM™ TRANSPARENT DRESSING, NEXCARE™, ADVANCED CURAD™, AQUA-PROTECT™, and modifications thereof. In some embodiments, examples of the covering include transdermal patches, such as DuoFilm®, Durageisc®, Butrans®, Evra®, etc.

Transdermal patches can be used as a cosmetic, topical, and/or transdermal delivery system (TDS). In some examples, a TDS can be designed to deliver a drug (e.g., a therapeutic drug) onto a subject's body (e.g., a patient's skin). In some cases, the drug can cross the skin of the patient into the bloodstream of the patient. The TDS can deliver a therapeutically effective amount of the drug. The TDS can include a protective layer, a drug carrier (e.g., a liquid, gel, or solid matrix, a membrane, a pressure sensitive membrane, etc.), and an adhesive to adhere to the patient's skin. In some cases, the drug carrier can be the adhesive.

Examples of the drug that can be delivered via the TDS and its therapeutic application include scopolamine for motion sickness, nitroglycerin for angina, clonidine for hypertension, and estradiol for female hormone replacement therapy. Other examples of the drug include, but are not limited to, methylphenidate, selegiline, rivastigmine, rotigotine, granisteron, buprenorphine, oestrodiol, fentanyl, nicotine, testosterone, etc. Other examples of therapeutic applications include, but are not limited to, attention deficit hyperactivity disorder, urologic issues, erectile dysfunction, dermatologic conditions, migraine, other types of acute and chronic cephalgia, Parkinson's disease, restless leg syndrome, pain management, etc.

Various components of the system (e.g., the carrier and/or the key device) disclosed herein can include or be fabricated from materials such as polyvinyl chloride, polyvinylidene chloride, low density polyethylene, linear low density polyethylene, polyisobutene, poly(ethylene-vinylacetate) copolymer, lightweight aluminum foil and combinations thereof, stainless steel alloys, commercially pure titanium, titanium alloys, silver alloys, copper alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, glass, and combinations thereof.

Various components of the system (e.g., the carrier and/or the key device) disclosed herein may have material composites, including one or more of the above materials, to achieve various desired characteristics such as strength, rigidity, elasticity, compliance, and/or durability. The components of the carrier and/or the key device, individually or collectively, may also be fabricated from a heterogeneous material such as a combination of two or more of the above-described materials. The components of the carrier and/or the key device may be monolithically formed or integrally connected.

An automated dispensing machine (ADM) as described herein may be a commercially available ADM including, for example the McLaughlin dispensing system, the Baxter ATC-212 dispensing system, and the Pyxis MedStation. In some embodiments, one or more of the carriers or key devices disclosed herein can be stored in a drawer of the ADM (e.g., a CUBIE pocket in the Pyxis MedStation).

A carrier as described herein can include a housing. A recess can be provided on a portion of the housing. A housing can include a casing, enclosure, shell, box, and the like. A housing can include one or more hollow chambers, cavities or recesses. The housing may be formed having any shape and/or size. The housing can be configured to support and store contents (e.g. medications) as described elsewhere herein.

A code as described herein may be a visual code. The term “visual code,” as used herein, can refer to optical, machine-readable, representation (e.g., marking) of data, where the data usually describes something about the article(s) carrying the visual code. In some cases, the article(s) may comprise one or more devices (e.g. the carrier or key device). The visual code can comprise one or more graphical visual elements (e.g., one or more pictorial and/or textual datagrams), including, but are not limited to, one-dimensional (1D) visual codes representing the data by varying the width or spacing of parallel lines, two-dimensional (2D) visual codes which represents the data as a geometrical pattern, such as Quick Response (QR) codes, and/or three-dimensional (3D) visual codes. In some cases, the 3D visual codes may be a layer comprising a plurality of 1D and/or 2D visual codes, or a plurality of 1D and/or 2D visual codes that are at different depths with respective to one another. The visual code may or may not be visible by the naked eye.

In some cases, the visual code can be read by a visual scanning system (e.g., a sensor), such that the visual scanning system can extract information (e.g., information about the article(s) carrying the visual code) stored in the visual code. In some cases, the visual code can be read by the visual scanning system, such that the visual scanning system can be operatively connected to an external database that contains such information. In some cases, the visual code can be read by a user.

In some embodiments, a code as described herein may be a non-visual code. For example, the code may include encrypted signals, locking/unlocking codes, electronic keys, cipher keys, authentication codes, etc.

C. Additional Aspects of Carrier

In some embodiments, the secure transport system as described herein can be utilized for transporting other contents, such as, for example, items related to medicine (e.g., nuclear medications, mail-order medications, medical supplies, etc.), diet (e.g., foods, mail-in meals), food industry (e.g., ingredients), military (e.g., confidential items such as confidential documents), police, banks (e.g., stock certificates, cash, wills, family heirlooms or keepsakes), etc. Additional examples of the contents can include jewelry, radioactive materials, furs, etc. In some cases, the secure transport system as described herein can be utilized as a “tubing system” for transporting mail or cash from point A (e.g., a register) to point B (e.g., an administrative office).

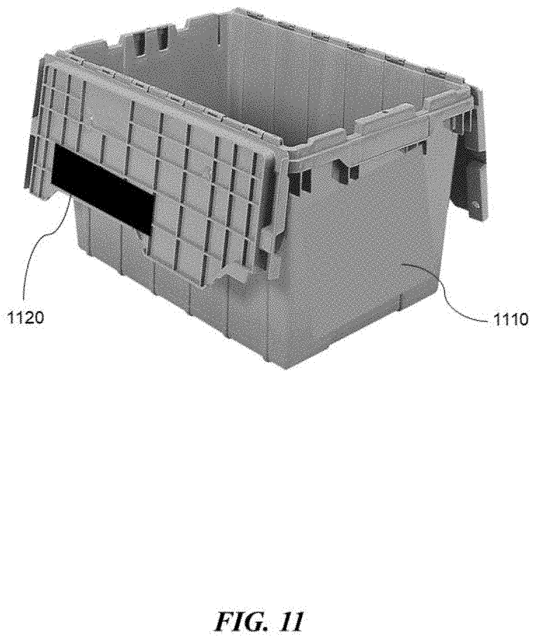

In some embodiments, the carrier as described herein can be a bin. FIG. 11 illustrates an example of a carrier 1110 for transporting the contents disclosed herein. The carrier 1110 can comprise a locking mechanism 1120 configured to lock the carrier and prevent any unauthorized access to the contents stored inside the carrier. The locking mechanism can be configured to be locked and/or unlocked by a key device, as described elsewhere herein. For example, the carrier 1110 can be used for distribution and delivery of medications to pharmacies in, for example, retail, hospital, nursing homes, hospices, etc.

In some embodiments, during the lifetime of the carrier (e.g., a carrier for transporting medications to a patient), the carrier can be configured to track and record (e.g., by generating a digital data) any individual (e.g., all individuals) having custody of the carrier, whether before, during, and/or after the delivery of the contents inside the carrier. For example, any individual responsible for having custody of the carrier during its lifetime can comprise a unique identification device. In order to hold, move, open, close, or work in any other means with the carrier, an individual can be required to hold the unique identification device to the carrier, such that the carrier can be configured to detect the unique key device (e.g., via wireless communications) and record an identity, time, and/or location at the time. The unique identification device can be similar to the key devices as described elsewhere herein. However, in some cases, the unique identification devices may not and need not be configured to unlock the carrier. Alternatively, the individual can scan a unique identifier of the carrier to record such identity, time, and/or location, and such scanning can generate the digital data and store it in a database operatively coupled to the carrier. In some cases, an individual may be responsible for loading the contents into the carrier. In some cases, an individual may be responsible for transporting the carrier to another destination without ever opening the carrier (e.g., a delivery courier personnel). Such individual can be a “mid-point” custodial party of the carrier and its contents, such as, for example, a clerk, a security guard, an airline attendant, etc. In some cases, an individual may be a final point of the supply chain, and the individual may be responsible for opening the carrier to receive the contents inside the carrier. As such, all individuals involved in the supply chain of the carrier can be tracked, recorded, and monitored. The carrier and/or the locking mechanism of the carrier can be operatively coupled to at least or up to 1, 2, 3, 4, 5, 6, 7, 8, 9, 10, 15, 20, 25, 30, 40, or 50 different unique identification devices.

In some embodiments, the systems and methods disclosed herein (e.g., the carriers) can be used to track distribution and/or delivery progress of the contents from an initial destination to a final destination. For example, the systems and methods can be used to track medications from a pharmacy to (i) a warehouse for storing the medications and/or (ii) a return center (e.g., a take-back system as described herein) for unused, leftover, or incorrectly delivered or prescribed medications. In another example, the systems and methods can be used to track delivery of any valuable items to be returned for a credit (e.g., a monetary credit). In another example, the systems can be used to track contents from a dispensing center (e.g., a warehouse) to paramedics and/or ambulances. In another example, the systems and methods can be used to track mail-order medications or returns thereof using a courier service (e.g., Amazon, UPS, FedEx, etc.).

In some embodiments, the carrier is disclosed herein can be configured to record information indicative of a surrounding of the carrier. The carrier can be configured to record the information when the carrier is opened, e.g., when the locking mechanism is unlocked. For example, the locking mechanism can comprise a tape to secure the carrier, and the carrier can be configured to record the information when at least a portion of the tape is removed. The information can be recorded in the form of a digital data that is stored in a database that is a part of the carrier or in communication with the carrier. In some cases, the information can be recorded and displayed on the carrier (e.g., on a display disposed on a surface of the carrier) in the form of a unique code. The unique code can be a machine readable code, a color code, a letter code, an image code, etc. The unique code can be displayed to display the occurrence of the carrier being at least partially opened. In some cases, the carrier can comprise one or more sensors (e.g., one or more cameras) configured to capture an image or a video of the surrounding when the carrier is at least partially opened. The one or more sensors can be disposed on an outer surface and/or an inner surface of the carrier. In some examples, the sensor can comprise one or more cameras disposed within the carrier to capture an image or video of a surrounding of the carrier when the carrier is opened.

In some embodiments, the carriers described herein can be configured to contain medications (e.g., unused, leftover, refused, and/or expired medications) while the medications are still in their original containers or packaging.

In some embodiments, the carrier can be used as a packaging for shipment of an item. The carrier can be programmed such that the carrier can be unlocked by a mailbox, a security/surveillance system of the destination (e.g., Ring Smart Home Security System of a recipient), or any other electronic device (e.g., a sensor embedded in a key device of the recipient or in a door of the recipient address) configured to communicate with the carrier. For example, when the carrier is delivered to the recipient and the carrier is placed inside the mailbox, carried across the security/surveillance system of the recipient, or is placed in the vicinity of the electronic device, a locking mechanism of the carrier may be unlocked such that the recipient can have access to the delivered item. Unlocking of the locking mechanism and/or opening of the carrier can trigger generation of data indicative of receipt of the item by the recipient.

In some embodiments, the carrier can be utilized to move medications from a dispensing area (e.g., a retail pharmacy) to the next destination (e.g., hospital, at-home patient, etc.). For example, once the prescriptions are ordered for a patient, the prescribed medications can be placed and locked in a carrier for pick-up. A clerk or the patient can have a key device that is configured (e.g., pre-programmed) to unlock the carrier to retrieve the prescribed medications.

In some embodiments, a delivery device (e.g., an autonomous vehicle, a robot, etc.) can be used to transport the carrier comprising a content from point A to point B. The delivery device can be configured to operate autonomously (e.g., global positioning system (GPS) navigated) or under remote control by a user. The delivery device can be configured to transport the carrier within a facility (e.g., within a floor or floor-to-floor within a building) or from one facility to another facility (e.g., between buildings). The delivery device can comprise a separate mechanism such that the carrier cannot be retrieved by an authorized user in the middle of the transport of the carrier to a target destination. The delivery device can comprise a sensor (e.g., a camera) configured to record information related to a surrounding of the delivery device during the delivery of the carrier to the target destination.

### IV. Blockchain

The database of the present disclosure to store information (e.g., time, date, location, and/or identity of a practitioner responsible for retrieving medication from an ADM, transporting the medication to the patient, administering of the medication by a caregiver to the patient, consumption of the medication by the patient, and the return/waste of unused or refused medication) for closed loop tracking of medications (e.g., prescription medications, non-prescription medications) can comprise or utilize a block chain (or “blockchain”) database. The term “blockchain,” as used herein, can refer to a suite of distributed ledger technologies that can be programmed to record and track anything of value (e.g., financial transactions, land titles, medical records, etc.). The blockchain can be a peer-to-peer (P2P) decentralized open ledger (or computer architecture thereof) that relies on a distributed network shared among its users. Each of the users can hold a public ledger of every transaction carried out using the architecture, and each public ledger can be checked against one another to ensure accuracy and accountability. Thus, a blockchain-based database (or blockchain database) can be used in place of a physical, centralized database, to record and handle one or more transactions of digital objects (e.g., data). Maintenance of the blockchain can be performed by a P2P network of communicating nodes (or computer systems) that are running a software. The software can be programmed with a specific application (e.g., cryptocurrency software, financial services software, supply chain software, smart contracts software, etc.). Transactions such as “party X transfers an object (e.g., a digital object, such as, for example, cryptocurrency, prescriptions, etc.) Y to party Z” can be broadcasted to the P2P network (e.g., by using one or more software applications). The network nodes can validate the transactions, add them to their copy of the ledger, and then broadcast these ledger additions to other nodes. Thus, the blockchain can be a distributed database, wherein, in order to independently verify the chain of ownership or validity of any and every transferred object, each network node stores its own copy of the blockchain. In some cases, a new group of transactions (i.e., a block) is created (e.g., at a predetermined frequency, such as, for example, 6 times per hour), added to the blockchain, and quickly published to all nodes in the P2P network. Thus, each block can contain a cryptographic hash of the previous block to keep the previous block “accountable.”

Tampering with transactions on the blockchain can become exponentially harder as time progresses, and can require extreme quantities of computing power to attempt, let alone succeed. In some cases, data stored in the blockchain can be included in integrity checks, in which transactions are assembled into a transaction merkle tree and hashed to produce a block header. Any alterations to transactions in a blockchain database can become apparent as the block would be invalid when indexed. As such, the blockchain's consensus mechanism can allow a data's hash to be published to the blockchain as irrefutable proof that the data existed at a given time in the past. Both the timestamp and the hash may be unalterable.

The device (e.g., the container, adapter, the key device, the medication programmed box, and/or the carrier as provided herein) can have an identifier (e.g., an identification device or a MRC). Scanning of such identifier may be updated to the blockchain database for closed loop tracking of medications, e.g., to track (i) supply, retrieval, transport, use, and return or waste of the contents (e.g. medications), (ii) personal linking (e.g., recording identification of practitioner(s) responsible for retrieving, transporting, and administering a drug to the patient), (iii) user or patient linking, (iv) pharmacy tracking, and/or (v) destroying the container containing the contents), etc. In an example, the blockchain database may provide a record (e.g., a permanent or irrefutable record) of each transaction as the valued contents (e.g., medications) are moved along the supply chain, to or within a hospital (e.g., in an ADM), to a device, to a user (e.g., a patient or caregiver), and back to a collection chain for discarding any unused medication. The blockchain database, as provided herein, can be an alterable and secured P2P network among patients, prescribers, pharmacy, government agencies (e.g., FDA, DEA, etc.), medication manufacturer, etc., to record and transfer data (e.g., medical history, prescription history, dates of prescription, retrieval, transport, administration, return, waste, etc.).

Although the present disclosure describes in detail the secure handling and tracking of medications (e.g., oral medications) for outpatient environments, it should be appreciated that the systems and methods described herein may be applicable to a variety of different field uses or end applications. For example, the disclosed systems and methods can be used for the secure handling and tracking of high value goods, disposal of dangerous, hazardous or toxic items, etc.

### V. Computer Systems

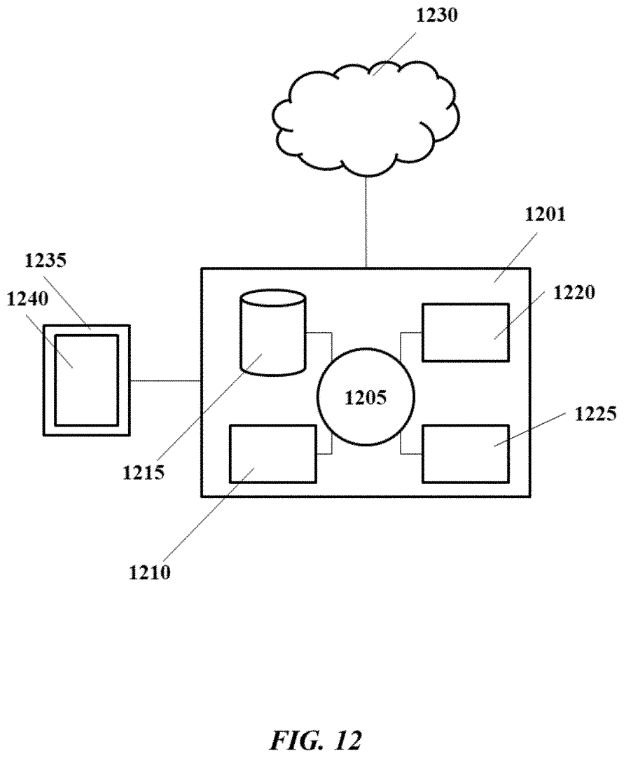

The present disclosure provides computer systems that are programmed to implement methods of the disclosure. FIG. 12 shows a computer system 1201 that is programmed or otherwise configured to track transport of one or more items (e.g., unused and/or leftover medications). The computer system 1201 can regulate various aspects of the transport system disclosed herein. The computer system 1201 can be an electronic device of a user or a computer system that is remotely located with respect to the electronic device. The electronic device can be a mobile electronic device.

The computer system 1201 includes a central processing unit (CPU, also “processor” and “computer processor” herein) 1205, which can be a single core or multi core processor, or a plurality of processors for parallel processing. The computer system 1201 also includes memory or memory location 1210 (e.g., random-access memory, read-only memory, flash memory), electronic storage unit 1215 (e.g., hard disk), communication interface 1220 (e.g., network adapter) for communicating with one or more other systems, and peripheral devices 1225, such as cache, other memory, data storage and/or electronic display adapters. The memory 1210, storage unit 1215, interface 1220 and peripheral devices 1225 are in communication with the CPU 1205 through a communication bus (solid lines), such as a motherboard. The storage unit 1215 can be a data storage unit (or data repository) for storing data. The computer system 1201 can be operatively coupled to a computer network (“network”) 1230 with the aid of the communication interface 1220. The network 1230 can be the Internet, an internet and/or extranet, or an intranet and/or extranet that is in communication with the Internet. The network 1230 in some cases is a telecommunication and/or data network. The network 1230 can include one or more computer servers, which can enable distributed computing, such as cloud computing. The network 1230, in some cases with the aid of the computer system 1201, can implement a peer-to-peer network, which may enable devices coupled to the computer system 1201 to behave as a client or a server.

The CPU 1205 can execute a sequence of machine-readable instructions, which can be embodied in a program or software. The instructions may be stored in a memory location, such as the memory 1210. The instructions can be directed to the CPU 1205, which can subsequently program or otherwise configure the CPU 1205 to implement methods of the present disclosure. Examples of operations performed by the CPU 1205 can include fetch, decode, execute, and writeback.

The CPU 1205 can be part of a circuit, such as an integrated circuit. One or more other components of the system 1201 can be included in the circuit. In some cases, the circuit is an application specific integrated circuit (ASIC).

The storage unit 1215 can store files, such as drivers, libraries and saved programs. The storage unit 1215 can store user data, e.g., user preferences and user programs. The computer system 1201 in some cases can include one or more additional data storage units that are external to the computer system 1201, such as located on a remote server that is in communication with the computer system 1201 through an intranet or the Internet.

The computer system 1201 can communicate with one or more remote computer systems through the network 1230. For instance, the computer system 1201 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1201 via the network 1230.

Methods as described herein can be implemented by way of machine (e.g., computer processor) executable code stored on an electronic storage location of the computer system 1201, such as, for example, on the memory 1210 or electronic storage unit 1215. The machine executable or machine readable code can be provided in the form of software. During use, the code can be executed by the processor 1205. In some cases, the code can be retrieved from the storage unit 1215 and stored on the memory 1210 for ready access by the processor 1205. In some situations, the electronic storage unit 1215 can be precluded, and machine-executable instructions are stored on memory 1210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1201 can include or be in communication with an electronic display 1235 that comprises a user interface (UI) 1240 for providing, for example, a UI on a display of the device disclosed herein (e.g., the container, adapter, the key device, the medication programmed box, and/or the carrier as provided herein) or a take-back system for returning of such device. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1205. The algorithm can, for example, (i) determine a probability of medication mismanagement (e.g., diversion) by a healthcare provider and/or (ii) determine a probability of mishandling of an item during its transport in the device disclosed herein.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-22. (canceled)

23. A system for secure transport of contents, the system comprising:
a carrier configured to receive and securely store the contents therein, wherein the carrier is configured to operate and switch between an unlocked state and a locked state, wherein the carrier is configured to display at least one code that is indicative of the unlocked state or the locked state, and wherein the carrier in the locked state is used by a user to transport the contents from a first location to a second location; and
a key device paired with the carrier, wherein the key device is configured to cause the carrier to switch from the locked state to the unlocked state upon activation of the key device, thereby permitting the user to access the contents from the carrier at the second location.

24. The system of claim 23, wherein the user is a healthcare practitioner, and the contents include medications or medical devices/equipment.

25. The system of claim 24, wherein the contents are administered by the healthcare practitioner to a patient at the second location.

26. The system of claim 25, wherein the key device is provided at the second location, and wherein the key device is activated when the carrier is brought in proximity to the key device at the second location.

27. The system of claim 26, wherein the key device is associated with a patient at the second location.

28. The system of claim 25, wherein the key device is provided at a third location remote to the second location, and wherein the key device is used to remotely unlock the carrier at the second location.

29. The system of claim 28, wherein the key device is associated with a third party at the third location, and wherein the third party is an advocate or proxy for the patient authorized to approve certain medical healthcare decisions for the patient.

30. The system of claim 26 or 28, wherein the key device and the carrier are in wireless communication with each other.

31. The system of claim 23, wherein the carrier is configured to display (i) a first code when the contents are secured in the locked carrier at the first location, wherein the first code is configured to be scanned by the user at the first location, or (ii) a second code when the carrier is unlocked at the second location and the contents accessed by the user, wherein the second code is configured to be scanned by the user at the second location.

32. A method for securing transport of contents, the method comprising:
(a) providing (i) a carrier configured to operate and switch between an unlocked state and a locked state, wherein the carrier is configured to display at least one code that is indicative of the unlocked state or the locked state, wherein the carrier in the locked state is used by a user to transport the contents from a first location to a second location and (ii) a key device paired with the carrier;
(b) using the carrier to receive and securely store the contents therein;
(c) subsequent to (b), activating the key device to cause the carrier to switch from the locked state to the unlocked state, thereby permitting the user to access the contents from the carrier at the second location.

33. The method of claim 32, wherein the user is a healthcare practitioner, and the contents include medications or medical devices/equipment.

34. The method of claim 33, wherein the contents are administered by the healthcare practitioner to a patient at the second location.

35. The method of claim 34, wherein the key device is provided at the second location, and the method further comprises activating the key device by bringing the carrier in proximity to the key device at the second location.

36. The method of claim 35, wherein the key device is associated with a patient at the second location.

37. The method of claim 34, wherein the key device is provided at a third location remote to the second location, and wherein the key device is used to remotely unlock the carrier at the second location.

38. The method of claim 37, wherein the key device is associated with a third party at the third location, and wherein the third party is an advocate or proxy for the patient authorized to approve certain medical healthcare decisions for the patient.

39. The method of claim 35 or 37, wherein the key device and the carrier are in wireless communication with each other.

40. The method of claim 32, wherein the carrier is configured to display (i) a first code when the contents are secured in the locked carrier at the first location, wherein the first code is configured to be scanned by the user at the first location, or (ii) a second code when the carrier is unlocked at the second location and the contents accessed by the user, wherein the second code is configured to be scanned by the user at the second location.

41. The system of claim 31, wherein the carrier is configured to display (i) the first code when the contents are secured in the locked carrier at the first location, wherein the first code is configured to be scanned by the user at the first location, and (ii) the second code when the carrier is unlocked at the second location and the contents accessed by the user, wherein the second code is configured to be scanned by the user at the second location.

42. The method of claim 32, wherein the carrier is configured to display (i) the first code when the contents are secured in the locked carrier at the first location, wherein the first code is configured to be scanned by the user at the first location, and (ii) the second code when the carrier is unlocked at the second location and the contents accessed by the user, wherein the second code is configured to be scanned by the user at the second location
